(19) United States
(12) Patent Application Publication
(10) Pub. No.: US 2026/0315504 A1
(43) Pub. Date: Oct. 15, 2026

(54) INORGANIC ION ELUTING NANOPARTICULATE MINERALIZED COLLAGEN GLYCOSAMINOGLYCAN MATERIALS

(71) Applicants: The Regents of the University of California, Oakland, CA (US); THE BOARD OF TRUSTEES OF THE UNIVERSITY OF ILLINOIS, Urbana, IL (US)

(72) Inventors: Justine C. Lee, Santa Monica, CA (US); Youngnam Kang, Los Angeles, CA (US); Wei Chen, Los Angeles, CA (US); Xiaoyan Ren, Los Angeles, CA (US); Meiwand Bedar, Los Angeles, CA (US); Brendan Harley, Urbana, IL (US)

(73) Assignees: The Regents of the University of California, Oakland, CA (US); THE BOARD OF TRUSTEES OF THE UNIVERSITY OF ILLINOIS, Urbana, IL (US)

(21) Appl. No.: 19/481,835

(22) PCT Filed: May 23, 2024
(86) PCT No.: PCT/US24/30748

§ 371 Date: Nov. 5, 2025

Related U.S. Application Data
(60) Provisional application No. 63/503,824, filed on May 23, 2023.

(51) Int. Cl. A61K 31/661; A61K 9/51; A61K 38/39; A61P 19/08

(52) U.S. Cl. CPC A61K 31/661; A61K 9/5146; A61K 9/5161; A61K 9/5169; A61K 38/39; A61P 19/08

(57) ABSTRACT

Compositions including a collagen glycosaminoglycan scaffold and a phosphate reservoir material are described. The compositions are useful in methods for promoting osteogenesis and attenuating bone resorption.

### CROSS-REFERENCE TO RELATED APPLICATION

This application claims the benefit under 35 U.S.C. Section 119(e) of co-pending and commonly-assigned U.S. Provisional Patent Application No. 63/503,824, filed May 23, 2023, the contents of which is incorporated by reference herein. This application is related to U.S. Patent Publication 20210052771, the entire contents of which are incorporated herein by reference.

### STATEMENT REGARDING FEDERALLY SPONSORED RESEARCH AND DEVELOPMENT

This invention was made with government support under DE028098 awarded by the National Institutes of Health. The government has certain rights in the invention. This work was supported by the U.S. Department of Veterans Affairs, and the Federal government has certain rights in the invention.

### TECHNICAL FIELD

The invention relates to materials and methods for promoting osteogenesis and attenuating bone resorption.

### BACKGROUND OF THE INVENTION

Coordination of bone formation and resorption is necessary for the success of bone regenerative strategies. Compositions which can serve as a template for bone growth while limiting bone resorption are needed for the treatment of trauma or congenital deformities affecting bone. The compositions and methods described herein satisfy this need.

### SUMMARY OF THE INVENTION

Studies of nanoparticulate mineralized collagen glycosaminoglycan (MC-GAG) materials provide evidence that MC-GAG is a promising base material for a cell-free, “off-the-shelf”, materials for inducing osteogenic signaling mechanisms in a variety of contexts including therapeutic interventions such as those used in calvarial regeneration. Building upon these discoveries, we have designed new C-CAG compositions that include selected phosphate releasing materials that can enhance osteogenic differentiation.

The invention disclosed herein has a number of embodiments. Embodiments of the invention include compositions comprising a collagen glycosaminoglycan scaffold coupled to one or more phosphate reservoir materials such as nanoparticles and/or hydrogels selected for their ability to release PO₄³⁻.into an aqueous environment in which the reservoir materials are disposed. Typically, the compositions of the invention are made by contacting a collagen glycosaminoglycan scaffold with a phosphate reservoir material such that the phosphate reservoir material coats or is disposed within the collagen glycosaminoglycan scaffold. In certain compositions of the invention, the collagen glycosaminoglycan scaffold is a nanoparticulate mineralized collagen glycosaminoglycan (MC-GAG) scaffold. In some embodiments of the invention, the compositions are designed to exhibit a phosphate release profile such that the concentration of PO₄³⁻ in a media in which the composition is disposed is at least 100 mM after 2, 3 or 4 days after the composition is disposed in the culture media (see, e.g., FIGS. 4, 6, 9 and 10). In some embodiments of the invention, the composition are designed to exhibit a phosphate release profile such that the concentration of PO₄³⁻ observed in the media is at least 10% or 25% higher after 2, 3 or 4 days in the culture media than concentrations of PO₄³⁻ observed in a control composition comprising the collagen glycosaminoglycan scaffold lacking the phosphate reservoir material.

In certain illustrative compositions of the invention, the phosphate reservoir material comprises nanoparticles which are disposed within a collagen glycosaminoglycan scaffold (e.g., nanoparticles formed using materials such as a beta-glycerophosphate and a polymeric material such as a polyethylene glycol). In other illustrative compositions of the invention, the phosphate reservoir material comprises a hydrogel coating disposed on the collagen glycosaminoglycan scaffold, wherein the hydrogel comprises phosphate moieties coupled thereto (e.g., a hydrogel comprising a chitosan and B-glycerophosphate). In some embodiments of the invention, the composition further comprises a therapeutic agent. Optionally, for example, the composition comprises a therapeutic protein such as osteoprotegerin (OPG). In certain embodiments of the invention, the composition further comprises a detectable marker or a pharmaceutically acceptable excipient.

Embodiments of the invention include methods of promoting osteogenesis in a subject in need thereof, comprising, or alternatively consisting essentially of, or yet consisting of, administering to the subject an effective amount of a composition, comprising, or alternatively consisting essentially of, or yet consisting of, a collagen glycosaminoglycan scaffold and one or more phosphate reservoir materials. In another aspect, provided are methods of attenuating bone resorption in a subject in need thereof, comprising or alternatively consisting essentially of, or yet consisting of, administering to the subject an effective amount of a composition comprising or alternatively consisting essentially of, or yet consisting of, a collagen glycosaminoglycan scaffold operatively coupled to one or more phosphate reservoir materials.

Embodiments of the invention include methods of inhibiting osteoclastogenesis in a subject in need thereof is provided, the method comprising, or alternatively consisting essentially of, or yet further consisting of administering to the subject an effective amount of the composition of any embodiment herein. In another aspect, a method of inhibiting osteoclast activation in a subject in need thereof is provided, comprising, or alternatively consisting essentially of, or yet further consisting of, administering to the subject an effective amount of the composition of any embodiment herein.

In another aspect, provided are methods of preparing a composition, the methods comprising or alternatively consisting essentially of, or yet consisting of, contacting a nanoparticulate mineralized collagen glycosaminoglycan (MC-GAG) scaffold with a solution comprising or alternatively consisting essentially of, or yet consisting of one or more phosphate reservoir materials. In another aspect, provided is a composition prepared by contacting a MC-GAG scaffold with a solution comprising one or more phosphate reservoir materials. One such embodiment of this method for making a composition of the invention comprises contacting a collagen glycosaminoglycan scaffold with a phosphate reservoir material such that the phosphate reservoir material coats and/or is disposed within the collagen glycosaminoglycan scaffold.

Yet further provided are kits comprising the compositions as described herein and instructions for use in vitro and/or in vivo.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Illustrative phosphate reservoir material (CS/β-GP Hydrogel) and associated methods. The top panel provides a cartoon schematic showing formation of the hydrogel using chitosan and β-glycerophosphate starting materials. The bottom panel provides an illustration of the methodology associated with this embodiment.

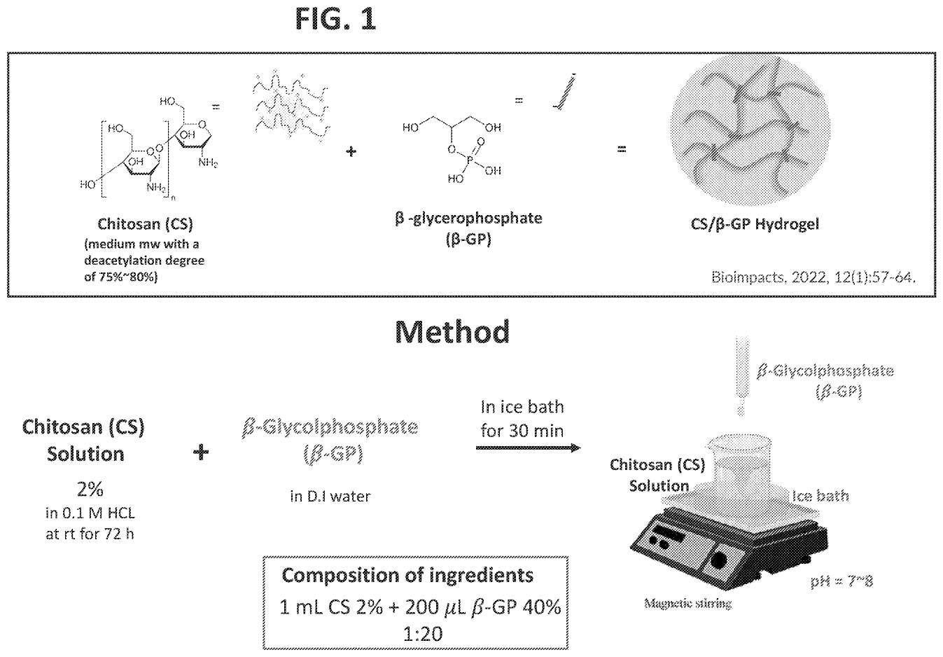

FIG. 2. ILLUSTRATIVE PHOSPHATE RESERVOIR MATERIALS INCLUDING INGREDIENTS, proportions and incubation times. This figure provides a number of photographs of embodiments of the invention having differing amounts of crosslinker and/or reaction incubation times.

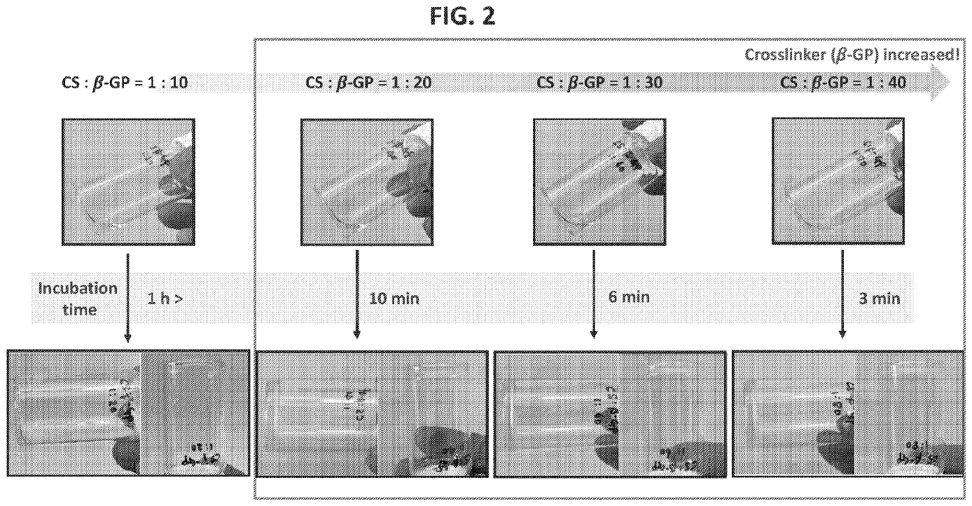

FIG. 3. Data From Studies of 2D CS/β-GP Hydrogel with cells (without MC-GAG). The left panel shows data from Alizarin red staining studies, the middle panel shows data from O.D. studies of various hydrogel phosphate reservoir embodiments, and the right panel shows data from phosphate release studies on one of these embodiments.

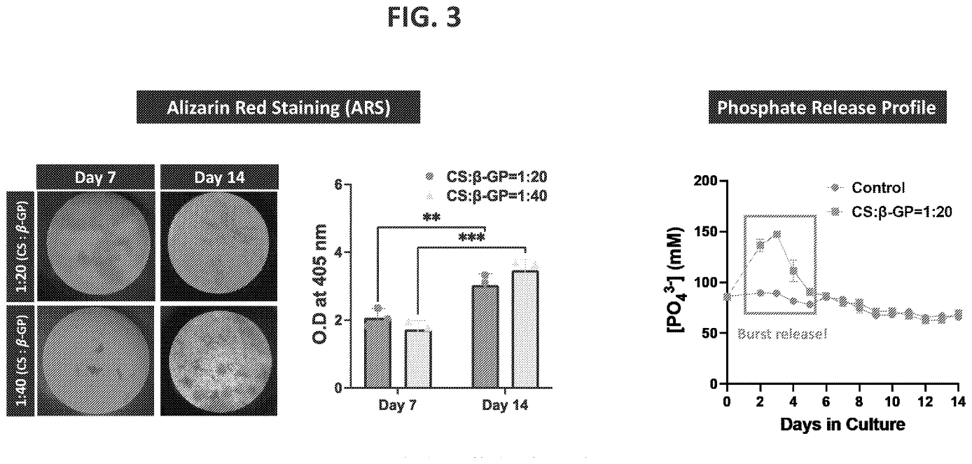

FIG. 4. Data From Studies of MC-GAG@CS/β-GP Hydrogel (MCGPh). The top panel provides a schematic of methods for making a phosphate reservoir hydrogel embodiment of the invention. The left bottom panel shows data from phosphate release studies on embodiments of the invention, the middle bottom panel shows data from Alizarin red staining studies on embodiments of the invention and the right panel shows data from O.D. studies on embodiments of the invention.

FIG. 5. Data From Studies of 3D CS/β-GP Hydrogels. The top panel shows data from methods of placing 3D CS/β-GP Hydrogel under MC as a phosphate supporting carrier. The left bottom panel shows data from stability studies of embodiments of the invention at day 0 and day 7 and the, the middle and right bottom panel show shows WST data from O.D. studies on embodiments of the invention.

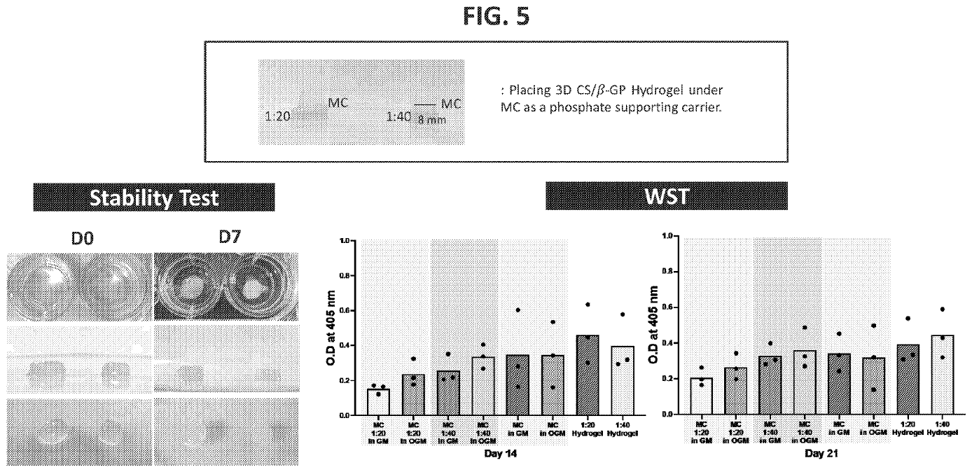

FIG. 6. Data from studies of an embodiment of the invention comprising compositions combined with a biomolecule in micelles. The left panel provides a schematic of 3D Vd@MCGPh hydrogel with cells, and the right panel shows data from phosphate release studies on embodiments of the invention.

FIG. 7. Data from studies of an embodiment of the invention comprising compositions combined with phosphate containing nanoparticles comprising β-GP molecules coupled together with a 4 arm PEG linking compound. The top panel shows formation of the hydrogel using polyethylene glycol (PEG), gelatin and β-glycerophosphate starting materials. The bottom panel shows different compositions of the invention and stiffness characteristics.

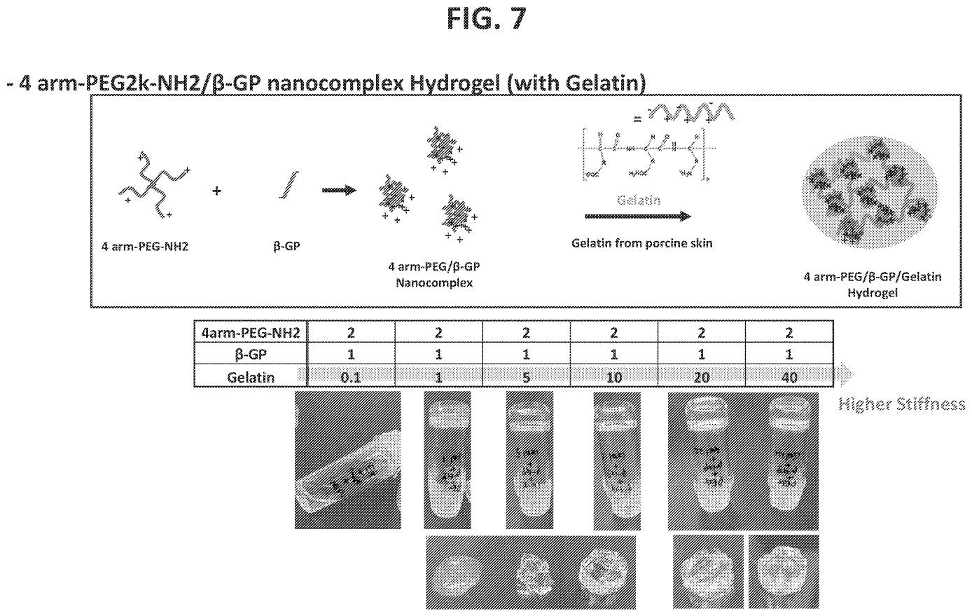

FIG. 8. Data from studies of another embodiment of the invention comprising compositions of the invention combined with a poly-D, L-lactic acid (PDLLA) plate. The left panels show photographs of a bone defect with only a PDLLA plate as well as with a PDLLA plate and compositions of the invention. The right panel shows photographs of bone regeneration after 6 months.

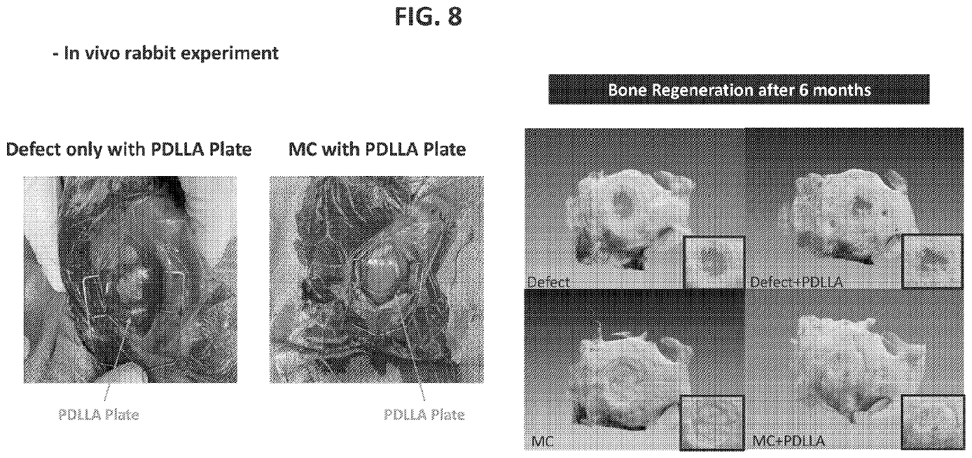

FIG. 9. Methods, assays and data from phosphate release studies. The right panel shows a schematic for the preparation of embodiments of the invention and the left panel shows the phosphate release profile of embodiments of the invention.

FIGS. 10A-10C. Assays and data from an invention embodiment comprising a CS/β-GP hydrogel. FIG. 10(A) show photos from studies of the stability of CS/β-GP hydrogel in PBS for 7 days in two different ratio (1:20 and 1:40). FIG. 10(B) provides an image of combination of MC-GAG and CS/β-GP hydrogel; Placing 3D CS/β-GP hydrogel under MC-GAG as a phosphate supporting carrier. FIG. 10(C) provides photographs of H&E and Alizarin Red Staining of MC-GAG, MC-GAG combined with CS/β-GP (1:20) group, and MC-GAG combined with CS/β-GP (1:40) group with hMSCs at day 14. FIG. 10(D) provides data from studies of cell viability of MC-GAG, MC-GAG combined with CS/β-GP (1:20) group, and MC-GAG combined with CS/β-GP (1:40) group with hMSCs at day 7, 14, and 21.

FIG. 11. Methods For Making MC-GAG@CS/β-GP nanoparticle materials. This figure provides a cartoon schematic of illustrative methods for making compositions where the phosphate reservoir comprises nanoparticles.

FIG. 12. Data From Studies of MC-GAG@CS/β-GP nanoparticle materials. The left panel provides DLS data from studies of an embodiments of the invention comprising about 10 nm-20 nm sized nanoparticles. The middle panel shows SEM images of such embodiments of the invention. The right panel shows data from phosphate elution studies of such embodiments of the invention.

FIG. 13. Data From Studies of MC-GAG@CS/β-GP nanoparticle materials. The left panel provides a series of live/dead assay images of cells combined with MC-GAG compositions where the phosphate reservoir comprises nanoparticles. The top right panel shows a series of images of Alizarin red stained cells combined with MC-GAG compositions where the phosphate reservoir comprises nanoparticles; and the bottom right panel shows graphed data on cell viability with cells combined with MC-GAG compositions where the phosphate reservoir comprises nanoparticles.

FIG. 14. Typical MC-GAG@CS/β-GP nanoparticle materials. The left panel provides a schematic of 4-ArmPEG5K-NH2/β-GP; the middle panel provides a schematic of 4-ArmPEG20K-NH2/β-GP; and the right panel provides a schematic of 8-ArmPEG20K-NH2/β-GP.

FIG. 15. Data From Studies of MC-GAG@CS/β-GP nanoparticle materials. This figure shows data from phosphate elution studies of nanoparticle embodiments shown in FIG. 14.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various publications, patents and published patent specifications are referenced by an identifying citation or by an Arabic numeral, the full citation of which is found preceding the claims. The disclosures of these publications, patents and published patent specifications are hereby incorporated by reference into the present disclosure in their entirety to more fully describe the state of the art to which this invention pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

13.8 million surgeries entering the skull occur annually for etiologies including trauma, stroke, cancer, and congenital anomalies. In each instance, skull reconstruction is required to prevent neurologic sequelae, cerebral protection, and potential psychosocial burdens particularly in large defects. The current clinically available materials for cranial defect reconstruction are limited by donor site morbidity for autologous bone and complications and cost for alloplastic materials, thereby providing an opportunity to develop strategies targeting skull regeneration. With the increasing knowledge of the instructive capabilities of the extracellular matrix, we previously demonstrated the potential for an extracellular matrix-inspired, synthetic material composed of nanoparticulate mineralized collagen glycosaminoglycan (MC-GAG) to serve as a materials-only regenerative strategy. MC-GAG was capable of regenerating massive calvarial defects in vivo without the addition of pre-expanded progenitor cells or exogenous growth factor supplementation. Mechanistically, we recently demonstrated that MC-GAG generates a temporospatial spike in phosphate ion equilibrium in the local microenvironment that is essential for its effects on osteoprogenitor cell differentiation via the activities of the type III sodium-phosphate cotransporters, SLC20A1/PiT-1 and SLC20A2/PiT-2. Building upon these studies, we determined that augmentation of phosphate elution improves MC-GAG-mediated osteoprogenitor differentiation. We can observe the effects of a composite material combining MC-GAG and a phosphate eluting hydrogel (MCGPh) on in vitro osteogenic differentiation of primary human mesenchymal stem cells. Our studies provide evidence that increasing the length of time for phosphate elution will improve osteogenic differentiation beyond that of the MC-GAG base material. Our studies provide evidence that increasing the length of time for phosphate elution will improve in vivo calvarial healing in a manner that will surpass that of MC-GAG alone.

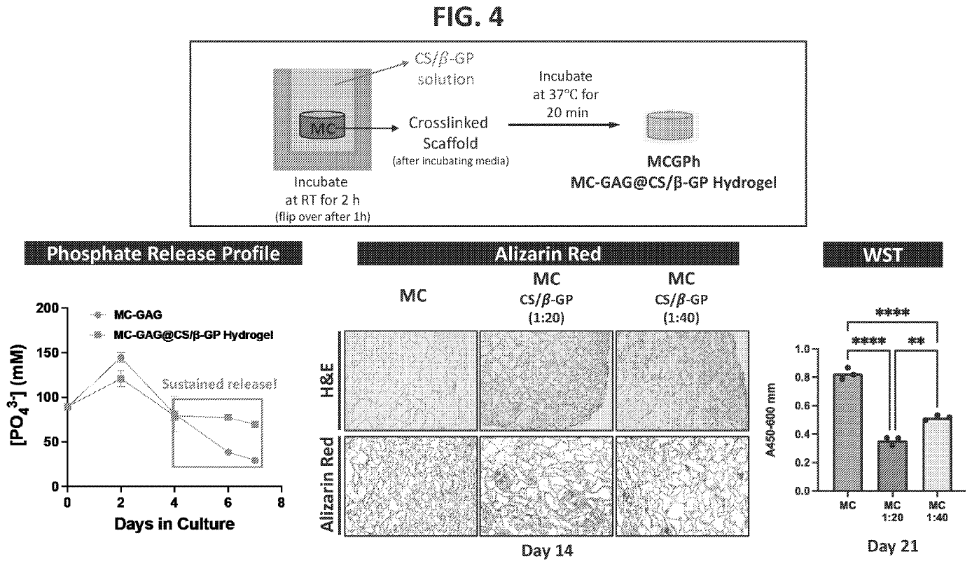

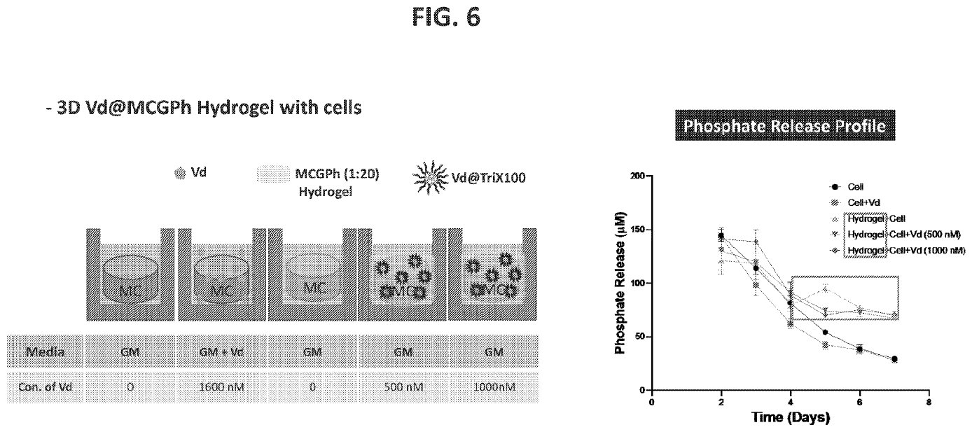

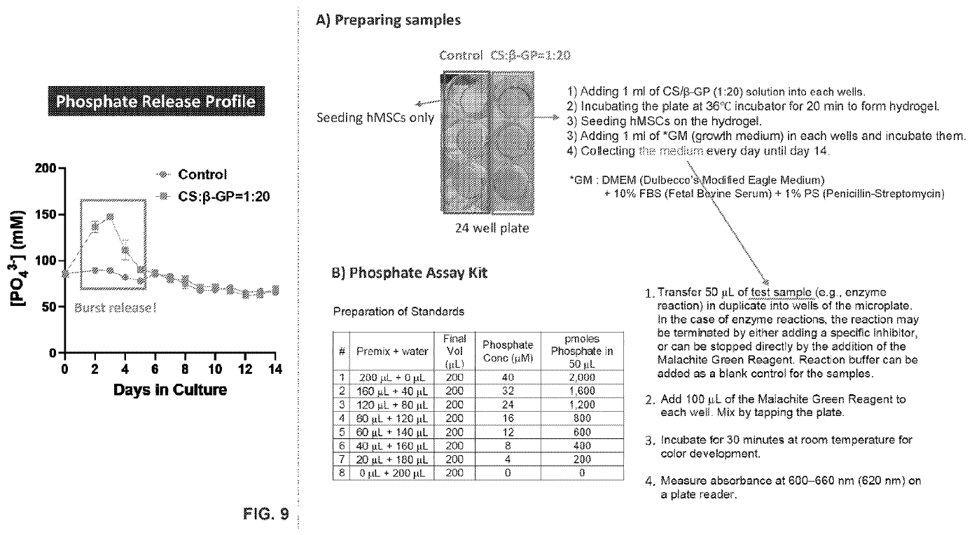

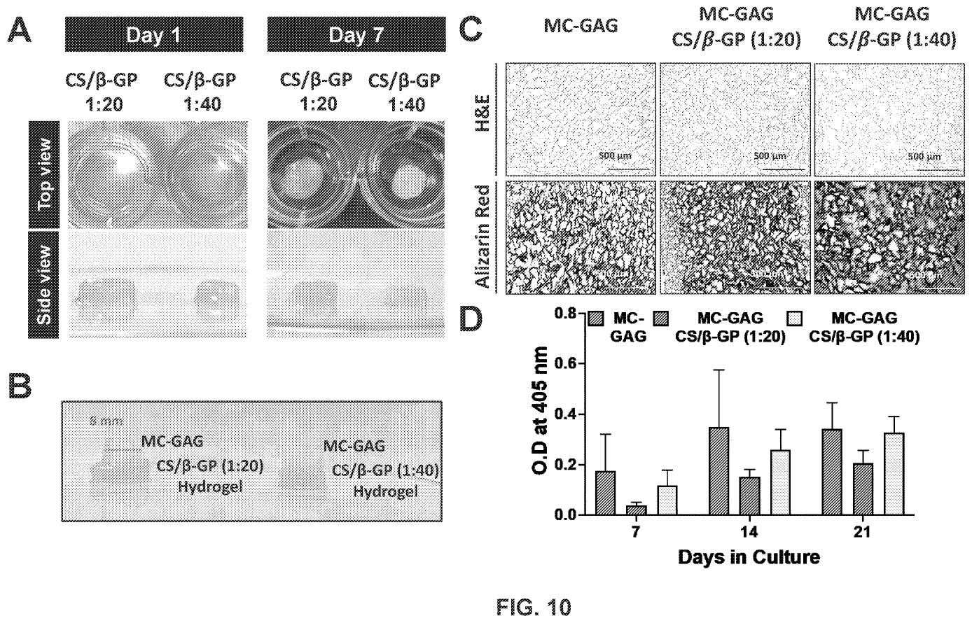

As discussed in detail below, embodiments of the invention include compositions comprising a collagen glycosaminoglycan scaffold coupled to one or more phosphate reservoir materials, and methods for making and using such compositions. Typically, the compositions of the invention are made by contacting a collagen glycosaminoglycan scaffold with a phosphate reservoir material such that the phosphate reservoir material coats and/or or is disposed within the collagen glycosaminoglycan scaffold. In certain compositions of the invention, the collagen glycosaminoglycan scaffold is a nanoparticulate mineralized collagen glycosaminoglycan (MC-GAG) scaffold. In some compositions of the invention, the phosphate reservoir material can comprise a hydrogel coating disposed on the collagen glycosaminoglycan scaffold, wherein the hydrogel comprises phosphate moieties coupled thereto (e.g., a hydrogel comprising a chitosan and β-glycerophosphate). In some embodiments of the invention, the compositions are designed to exhibit a phosphate release profile such that the concentration of PO₄³⁻ in a media in which the composition is disposed is at least 100 mM after 2, 3 or 4 days after the composition is disposed in the culture media (see, e.g., FIGS. 4, 6, 9 and 10). In some embodiments of the invention, the composition are designed to exhibit a phosphate release profile such that the concentration of PO₄³⁻ observed in the media is at least 10% or 25% higher after 2, 3 or 4 days in the culture media than concentrations of PO₄³⁻ observed in a control composition comprising the collagen glycosaminoglycan scaffold lacking the phosphate reservoir material.

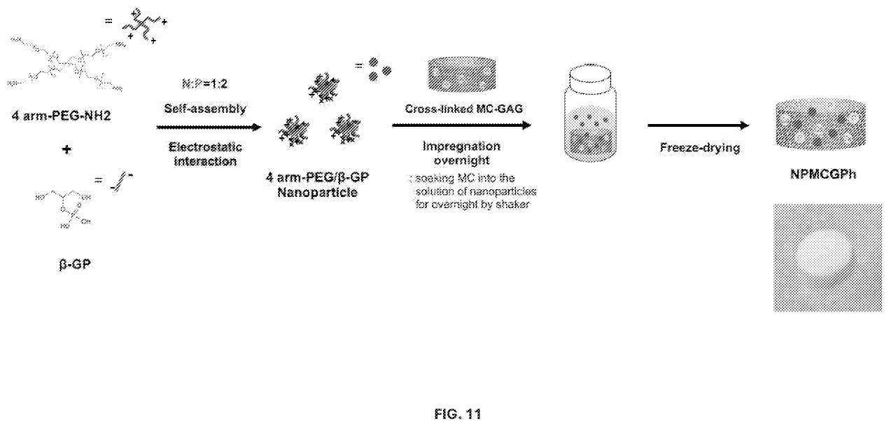

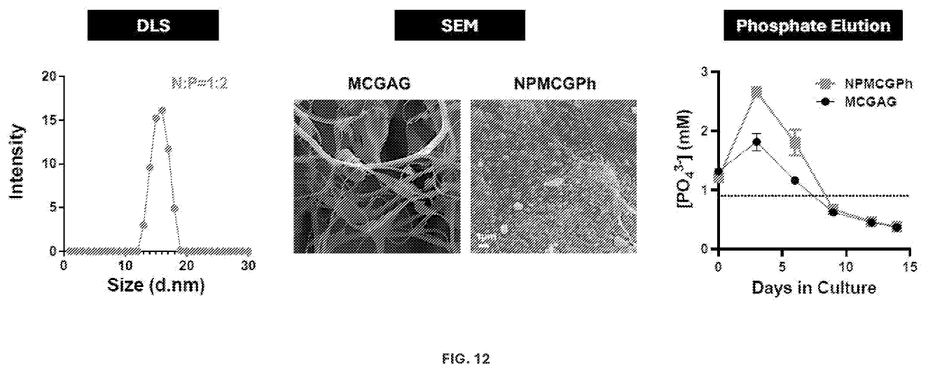

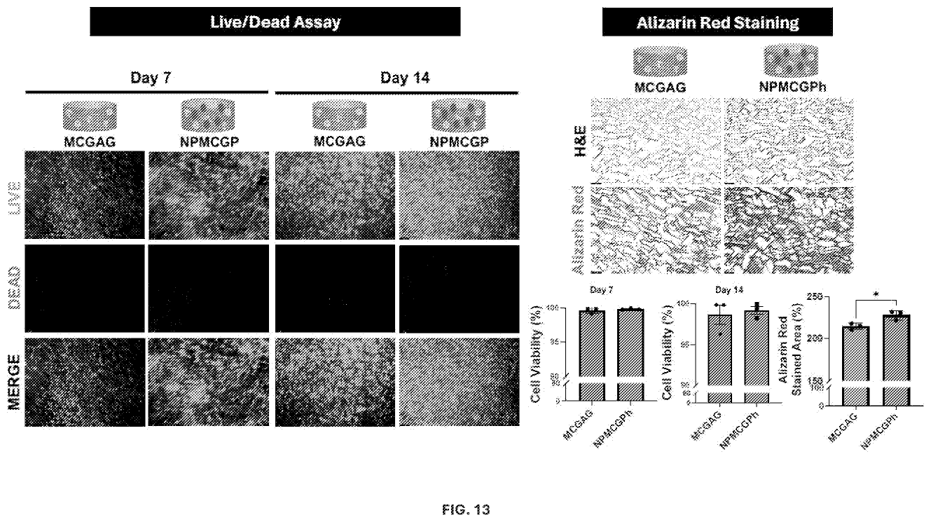

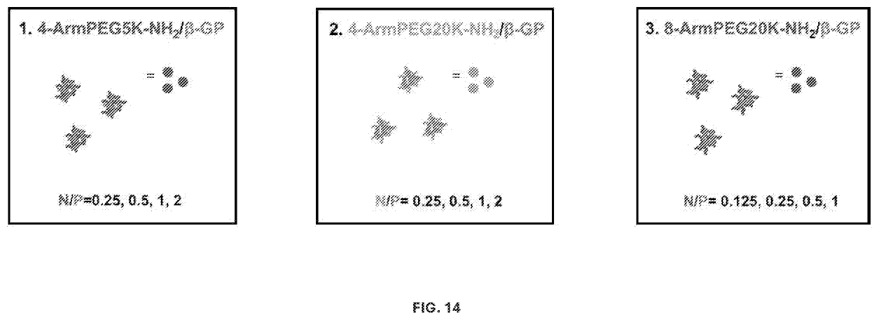

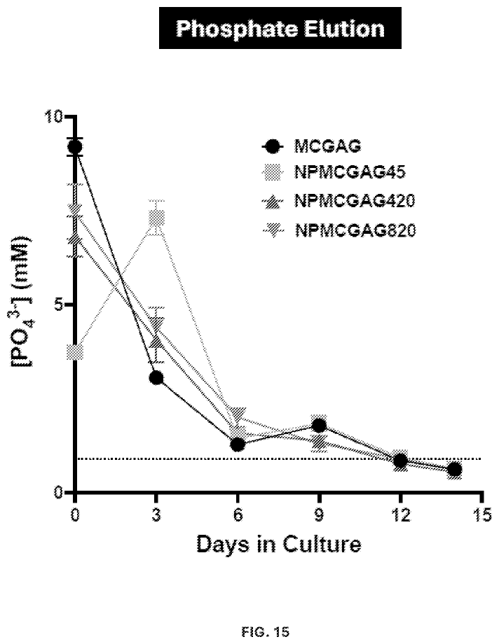

In certain compositions of the invention, the phosphate reservoir material can comprise nanoparticles which are disposed or entrapped within, or and/or crosslinked to a collagen glycosaminoglycan scaffold (e.g., nanoparticles formed using materials such as a beta-glycerophosphate and a polymeric material such as a polyethylene glycol). In some embodiments of the invention, the nanoparticles are designed/formed exhibit an average diameter of at least 10, 50, 100, 250 or 500 nm. In certain embodiments, the nanoparticles are designed/formed exhibit an average diameter of 100 nm to 600 nm. In typical embodiments of the invention, the polymeric material is designed to comprise a charge selected to counterbalance the charge of β-GP molecules (e.g. a PEG polymeric material comprising a charged NH2 group) so that the net charge of the nanoparticles in neutral. In this context, certain embodiments of the invention use selected ratios of polymers to β-GP molecules to modulate the charge of the nanoparticles in this way. Illustrative working examples of such nanoparticle phosphate reservoir material embodiments of the invention are shown in FIGS. 11-15. For example, FIG. 14 shows schematics of 4 arm-PEG/β-GP and 8 arm-PEG/β-GP embodiment. FIGS. 11-15 further show data from studies on these embodiments of the invention. In some embodiments of the invention, the composition further comprises a therapeutic agent. Optionally, for example, the composition comprises a therapeutic protein such as osteoprotegerin (OPG). In certain embodiments of the invention, the composition further comprises a pharmaceutically acceptable excipient.

Embodiments of the invention include methods of promoting osteogenesis in a subject in need thereof, comprising, or alternatively consisting essentially of, or yet consisting of, administering to the subject an effective amount of a composition, comprising, or alternatively consisting essentially of, or yet consisting of, a collagen glycosaminoglycan scaffold and one or more phosphate reservoir materials. In another aspect, provided are methods of attenuating bone resorption in a subject in need thereof, comprising or alternatively consisting essentially of, or yet consisting of, administering to the subject an effective amount of a composition comprising or alternatively consisting essentially of, or yet consisting of, a collagen glycosaminoglycan scaffold operatively coupled to one or more phosphate reservoir materials.

Embodiments of the invention include methods of inhibiting osteoclastogenesis in a subject in need thereof is provided, the method comprising, or alternatively consisting essentially of, or yet further consisting of administering to the subject an effective amount of the composition of any embodiment herein. In another aspect, a method of inhibiting osteoclast activation in a subject in need thereof is provided, comprising, or alternatively consisting essentially of, or yet further consisting of, administering to the subject an effective amount of the composition of any embodiment herein.

In another aspect, provided are methods of preparing a composition, the methods comprising or alternatively consisting essentially of, or yet consisting of, contacting a nanoparticulate mineralized collagen glycosaminoglycan (MC-GAG) scaffold with a solution comprising or alternatively consisting essentially of, or yet consisting of one or more phosphate reservoir materials. In another aspect, provided is a composition prepared by contacting a MC-GAG scaffold with a solution comprising one or more phosphate reservoir materials. One such embodiment of this method for making a composition of the invention comprises contacting a collagen glycosaminoglycan scaffold with a phosphate reservoir material such that the phosphate reservoir material coats the collagen glycosaminoglycan scaffold. Yet further provided are kits comprising the compositions as described herein and instructions for use in vitro and/or in vivo.

“Glycosaminoglycan” as used herein, intends a polysaccharide comprising a repeating disaccharide unit which comprises an amino sugar and an uronic sugar. “Collagen” as used herein, refers to the main structural protein of the extracellular space in the connective tissues of animal bodies comprising amino acids wound together to form triple-helices to form elongated fibrils. “Type I collagen” as used herein, intends a type of collagen that forms large eosinophilic fibers known in the art as collagen fibers. “Phosphate reservoir material” as used herein refers to materials selected to release phosphate (PO₄³⁻ ) ions into an aqueous environment in which the material is disposed. As shown in the examples, below illustrative phosphate reservoir materials include phosphate eluting hydrogels.

“Nanoparticulate mineralized collagen glycosaminoglycan (MC-GAG) scaffold” as used herein, intends a scaffold that is a substrate for bone regrowth. The scaffold may be used to repair cranial defects and trauma by placement at the site of defect or injury and stimulating bone regeneration at the site. MC-GAG scaffolds may be prepared as known in the art (see, e.g. U.S. Patent Publication 20210052771) or prepared according to methods disclosed herein. “Collagen glycosaminoglycan scaffold” as used herein refers to scaffolds including, but not limited to a MC-GAG scaffold or a Col-GAG scaffold. Collagen glycosaminoglycan scaffolds can be prepared using a lyophilization process of collagen and glycosaminoglycans (GAGs) or collagen-glycosaminoglycan-calcium phosphate produced by combining microfibrillar, type I collagen (Collagen Matrix, Oakland, N.J.) and chondroitin-6-sulfate (Sigma-Aldrich, St. Louis, Mo.) in a solution of 0.005 M to 0.1 M acetic acid, preferably 0.05 M acetic acid (pH 3.2) or with calcium salts not limited to calcium nitrate hydrate: Ca(NO₃)₂4H₂O or calcium hydroxide: Ca(OH)₂, Sigma-Aldrich) in a solution of phosphoric acid.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Administration,” “administering” intends local or systemic administration. In one aspect, local administration is surgical implantation of the compositions described herein. Administration may be accomplished implanting the composition directly or coating or impregnating a surgical implant or prosthesis with the compositions of the disclosure. The compositions may be implanted anywhere throughout the body of the subject where the growth or regeneration of bone is needed. Non-limiting examples include the skull, the facial bones or other bones, large or small in the subject.

It is to be understood that the terms “subject” and “patient” are interchangeable. An animal, subject or patient for diagnosis or treatment refers to an animal such as a mammal, or a human, ovine, bovine, feline, canine, equine, simian, etc. Non-human animals subject to diagnosis or treatment include, for example, simians, murine, such as, rat, mice, canine, leporid, livestock, sport animals, and pets. In one aspect, the subject is a human. “Scaffold” as used herein, intends a three dimensional analog of the extracellular matrix.

Administration or treatment in “combination” refers to administering two agents such that their pharmacological and/or therapeutic effects are manifest at the same time. Combination does not require administration at the same time or substantially the same time, although combination can include such administrations.

An “effective amount” is an amount sufficient to effect beneficial or desired results. An effective amount can be administered in one or more administrations or applications. Such delivery is dependent on a number of variables including the time period for which the individual composition is to be used, the bioavailability of the therapeutic agents included with the composition, the route of administration, etc. It is understood, however, that specific dose levels of the additional therapeutic agents disclosed herein for any particular subject depends upon a variety of factors including the activity of the specific compound employed, bioavailability of the compound, the route of administration, the age of the animal/subject and its body weight, general health, sex, the diet of the animal/subject, the time of administration, the rate of excretion, the drug combination, and the severity of the particular disorder being treated and form of administration. These considerations, as well as effective formulations and administration procedures are well known in the art and are described in standard textbooks. Consistent with this definition and as used herein, the term “therapeutically effective amount” is an amount sufficient to treat a specified disorder or disease or alternatively to obtain a pharmacological response such as immunosuppression, osteogenesis, bone resorption or mineralization.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease or trauma), stabilized (i.e., not worsening) state of a condition (including disease or trauma), delay or slowing of condition (including disease or trauma), progression, amelioration or palliation of the condition (including disease or trauma), states and remission (whether partial or total), whether detectable or undetectable. In one aspect, the term “treatment” excludes prevention or prophylaxis.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a therapeutic protein (e.g. osteoprotegerin), antibody, polypeptide or nucleic acid, intends those having minimal homology while still maintaining desired structure or functionality similar to the reference protein, antibody, polypeptide or nucleic acid. Unless specifically recited herein, it is contemplated that any polynucleotide, polypeptide or protein mentioned herein also includes equivalents thereof. For example, an equivalent intends at least about 70% homology or sequence identity, or at least 80% homology or sequence identity and alternatively, or at least about 85%, or alternatively at least about 90%, or alternatively at least about 95%, or alternatively 98% percent homology or sequence identity and exhibits substantially equivalent biological activity to the reference protein, antibody, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

As used herein, “homology” or “identical”, percent “identity” “sequence identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. The terms “homology” or “identical”, percent “identity” “sequence identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Exogenous,” “exogenously” and the like are intended to describe a material that is present and active in an organism or cell but that originated outside that organism or cell. “Endogenous,” “endogenously” and the like, as used herein, describes a protein that originates from the present cell or organism.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

“Osteoblast” as used herein, refers to a cell which is the major cellular component of bone with a single nucleus that synthesizes bone. Osteoblasts are specialized, terminally differentiated products of mesenchymal stem cells. Osteoblasts synthesize dense, cross-linked collagen, osteocalcin, and osteopontin. Osteoblasts mineralize the majority of the bone matrix in air breathing vertebrates.

As used herein, “osteocyte” refers to an osteoblast which is buried within the bone matrix. Osteocytes are the most commonly found cell in mature bone tissue. Osteocytes have a stellate shape, approximately 7 micrometers deep and wide by 15 micrometers in length. The cell body varies in size from 5-20 micrometers in diameter and contains 40-60 cell processes per cell, with a cell to cell distance between 20-30 micrometers. A mature osteocyte contains a single nucleus that is located toward the vascular side and has one or two nucleoli and a membrane. The cell also exhibits a reduced size endoplasmic reticulum, Golgi apparatus and mitochondria, and cell processes that radiate towards the mineralizing matrix. Osteocytes form an extensive connecting syncytial network via small cytoplasmic/dendritic processes in canaliculi.

As used herein, “osteogenesis” intends formation of bone, and is meant to include both natural and artificial means of bone formation. As used herein, “bone resorption” or “resorption” intends the process by which osteoclasts break down the tissue in bones and release minerals.

As used herein, “harvest” intends removal of biological material from the subject. A non-limiting example of harvesting biological material is harvesting stem cells. Stem cells may be harvested from a subject for either autologous or allogenic use in the same or different subject. Harvest of stem cells can be accomplished by methods known to the skilled artisan. Non-limiting examples of doing so include harvest from bone marrow or harvest from peripheral blood.

As used herein, “seeding” intends incorporation or infusion of mesenchymal stem cells (MSCs) into and/or onto a collagen glycosaminoglycan scaffold (MC-GAG or Col-GAG scaffold). Seeding can be accomplished using techniques known to the skilled artisan, including, but not limited to placement of a suspension of mesenchymal stem cells in growth media and pipetting this mixture onto the scaffold.

As used herein, “medium” refers to a growth medium or culture medium that is a solid, liquid or semi-solid designed to support the growth of cells. “Differentiation medium” refers to a medium specifically for inducing differentiating of an MSC. Non-limiting examples of components of a differentiation medium for MSCs include, fetal-bovine serum, penicillin-streptomycin, glutamine, β-glycerophosphate, ascorbic acid, and dexamethasone.

A preservative or cryoprotectant can be combined or admixed with the cells, scaffolds, nucleic acids and proteins or compositions containing them. These compositions can be lyophilized using methods known in the art and/or formulated into appropriate dosage forms for ease of use. As used herein, “cryoprotectant” intends a substance used to protect biological tissue from freezing damage. Non-limiting examples include sugars, glycols, dimethyl sulfoxide, and trehalose.

Materials inspired by bone-specific extracellular matrix (ECM) components, such as the nanoparticulate mineralized collagen glycosaminoglycan scaffold have generated great enthusiasm in regenerative technologies due to their abilities to instruct osteoprogenitor differentiation. Nanoparticulate mineralized collagen glycosaminoglycan material (MC-GAG) induces efficient mineralization of bone marrow-derived primary human mesenchymal stem cells (hMSCs) and primary rabbit bone marrow stromal cells (rBMSCs) in a manner that required an autogenous activation of the bone morphogenetic protein receptor (BMPR) signaling pathway through phosphorylation of small mothers against decapentaplegic-1/5 (Smad1/5). Furthermore, MC-GAG induces in vivo rabbit calvarial regeneration without the addition of exogenous growth factors or progenitor cells.

In osteoclast regulation, MC-GAG demonstrated both direct and indirect inhibitory effects on osteoclast viability, proliferation, and activation. In comparison to its non-mineralized collagen glycosaminoglycan (Col-GAG) counterpart, MC-GAG also induces hMSCs to express higher levels of osteoprotegerin early in osterogenic differentiation via intracellular signaling pathways distinct from those governing osteogenic differentiation.

### Compositions

In one aspect, provided are compositions comprising, or consisting essentially of, or yet further consisting of a collagen glycosaminoglycan scaffold in combination with a selected phosphate reservoir material. In a further aspect, the compositions are combined with a carrier, such as a pharmaceutically acceptable carrier, and optionally a cryoprotectant or preservative. The compositions can be formulated and lyophilized or frozen for ease of storage and use. In addition, they can be provided in specific dosages for ease of administration.

In some embodiments, the collagen glycosaminoglycan scaffold is a nanoparticulate mineralized collagen glycosaminoglycan (MC-GAG) scaffold. In some embodiments, the collagen glycosaminoglycan scaffold is a non-mineralized collagen glycosaminoglycan (Col-GAG) scaffold. In some embodiments, the collagen is type I collagen. In some embodiments, the collagen glycosaminoglycan scaffold comprises a porosity of about 10%, 15% 20%, 25%, 30%, 35%, 40%, 45%, 50%, 55%, 60%, 65%, 70%, 75%, 80%, 85%, 90%, or 95%. In some embodiments the collagen glycosaminoglycan scaffold comprises a porosity of about 40%. In some embodiments the collagen glycosaminoglycan scaffold comprises a porosity of about 45%. In some embodiments the collagen glycosaminoglycan scaffold comprises a porosity of about 50%. In some embodiments the collagen glycosaminoglycan scaffold comprises a porosity of about 55%. In some embodiments the collagen glycosaminoglycan scaffold comprises a porosity of about 60%. In some embodiments the collagen glycosaminoglycan scaffold comprises a porosity of about 65%. In some embodiments the collagen glycosaminoglycan scaffold comprises a porosity of about 70%. In some embodiments the collagen glycosaminoglycan scaffold comprises a porosity of about 75%. In some embodiments the collagen glycosaminoglycan scaffold comprises a porosity of about 80%. In some embodiments the collagen glycosaminoglycan scaffold comprises a porosity of about 85%. In some embodiments the collagen glycosaminoglycan scaffold comprises a porosity of about 90%. In some embodiments the collagen glycosaminoglycan scaffold comprises a porosity of about 95%.

In some embodiments the collagen glycosaminoglycan scaffold comprises a pore size between about 5 μm to about 10 μm. In some embodiments the collagen glycosaminoglycan scaffold comprises a pore size between about 10 μm to about 40 μm. In some embodiments the collagen glycosaminoglycan scaffold comprises a pore size between about 40 μm to about 70 μm. In some embodiments the collagen glycosaminoglycan scaffold comprises a pore size between about 70 μm to about 100 μm. In some embodiments the collagen glycosaminoglycan scaffold comprises a pore size between about 100 μm to about 130 μm. In some embodiments the collagen glycosaminoglycan scaffold comprises a pore size between about 130 μm to about 160 μm. In some embodiments the collagen glycosaminoglycan scaffold comprises a pore size between about 160 μm to about 190 μm. In some embodiments the collagen glycosaminoglycan scaffold comprises a pore size between about 210 μm to about 240 μm. In some embodiments the collagen glycosaminoglycan scaffold comprises a pore size greater than 240 μm.

In some embodiments the morphology of the scaffold comprises isotropic pores with a transverse: longitudinal pore aspect ratio of about 0.05. In some embodiments the morphology of the scaffold comprises isotropic pores with a transverse: longitudinal pore aspect ratio of about 0.15. In some embodiments collagen glycosaminoglycan scaffold comprises isotropic pores with a transverse: longitudinal pore aspect ratio of about 0.25. In some embodiments the morphology of the scaffold comprises isotropic pores with a transverse: longitudinal pore aspect ratio of about 0.35. In some embodiments collagen glycosaminoglycan scaffold comprises isotropic pores with a transverse: longitudinal pore aspect ratio of about 0.45. In some embodiments the collagen glycosaminoglycan scaffold comprises isotropic pores with a transverse: longitudinal pore aspect ratio of about 0.55. In some embodiments the collagen glycosaminoglycan scaffold comprises isotropic pores with a transverse: longitudinal pore aspect ratio of about 0.65. In some embodiments the collagen glycosaminoglycan scaffold comprises isotropic pores with a transverse: longitudinal pore aspect ratio of about 0.75. In some embodiments the collagen glycosaminoglycan scaffold comprises isotropic pores with a transverse: longitudinal pore aspect ratio of about 0.85. In some embodiments the collagen glycosaminoglycan scaffold comprises isotropic pores with a transverse: longitudinal pore aspect ratio of about 0.95. In some embodiments the collagen glycosaminoglycan scaffold comprises isotropic pores with a transverse: longitudinal pore aspect ratio of about 0.99.

Compositions comprising the compounds described herein can be manufactured by means of conventional mixing, dissolving, granulating, dragee-making levigating, emulsifying, encapsulating, entrapping, or lyophlization processes. The compositions can be formulated in conventional manner using one or more physiologically acceptable carriers, diluents, excipients, or auxiliaries which facilitate processing of the compounds provided herein into preparations which can be used in vitro or in vivo.

In some embodiments, the composition further comprises, or consists essentially of, or yet further consist of, a carrier. In some embodiments, the carrier further comprises one or more of a cryoprotectant or a preservative.

In another aspect, provided is a composition prepared by contacting a MC-GAG scaffold with a solution comprising a phosphate reservoir material. In some embodiments, the solution comprises MC-GAG combined with a hydrogel and/or nanoparticle formed from a polymer coupled to phosphate moieties.

In another aspect, provided are methods of promoting osteogenesis in a subject in need thereof, the methods comprising, or alternatively consisting essentially of, or yet further consisting of, administering to the subject an effective amount of a composition comprising a collagen glycosaminoglycan scaffold combined with a phosphate reservoir material and optionally another therapeutically active agent such as a protein such as osteoprotegerin (OPG), an OPG fragment or an equivalent of each thereof. As used herein and unless specifically noted otherwise, OPG intends full length or a fragment of the protein, as well as mammalian OPG and biological equivalents thereof. Effective amounts can be determined by the treating physician or veterinarian, and will vary with the subject being treated, the composition being used and the indication.

In another aspect, provided are methods of attenuating bone resorption in a subject in need thereof, the methods comprising, or alternatively consisting essentially of, or yet further consisting of, administering to the subject an effective amount of a composition as described herein. In some embodiments, the subject is a mammal. In some embodiments, the subject is a human.

In one aspect, provided are methods of preparing a composition comprising contacting a MC-GAG scaffold with a solution comprising a phosphate reservoir material. Embodiments of the invention include methods that add an additional phosphate eluting hydrogel or nanoparticle to the MC-CAG material to increase its osteogenic capabilities. Illustrative embodiments include (1) the addition of a chitosan/beta glycerophosphate hydrogel to nano particulate mineralized collagen glycosaminoglycan material; (2) the addition of PEGylated-phosphate or PEGylated-beta glycerophosphate to nanoparticulate mineralized collagen glycosaminoglycan material; (3) Addition of a hydrogel composed of chitosan with PEGylated-phosphate or PEGylated-beta glycerophosphate to nanoparticulate mineralized collagen glycosaminoglycan material. As will be appreciated by those of skill in this technology, depending on the phosphate release kinetics and quantities, any of these options may be modified to generate phosphate reservoirs with selected properties.

In one embodiment, the composition comprises, or alternatively consists essentially of, or yet further consists of three or more of a MC-GAG scaffold material, a phosphate reservoir material, an imaging agent, and a therapeutic agent such as OPG non-covalently incorporated therein. In one embodiment the method comprises, consists essentially or, or yet further consists of lyophilizing a suspension comprising, or alternatively consisting essentially of, or yet further consisting of microfibrillar type I collagen and chondroitin-6-sulfate in a solution comprising, or alternatively consisting essentially of, or yet further consisting of acetic acid. In some embodiments, the solution further comprises, or alternatively consists essentially of, or yet further consists of a therapeutic polypeptide or a fragment thereof. In some embodiments, the solution lyophilized does not comprise, or alternatively consist essentially of, or yet further consist of a therapeutic polypeptide or a fragment thereof and the process further comprises, or alternatively consists essentially of, or yet further consists of freezing the solution and sublimating the frozen solution to produce a scaffold, contacting the scaffold with a solution comprising, consisting essentially of, or consisting of 1-ethyl-3-(3-dimethylaminopropyl) carbodiimide and N-hydroxysuccinimide, and contacting the scaffold with a solution comprising, or alternatively consisting essentially of, or yet further consisting of a phosphate reservoir material or a therapeutic polypeptide or a fragment thereof.

In some embodiments, the MC-GAG scaffold is sterilized. In some embodiments, the MC-GAG scaffold is sterilized with ethylene oxide and crosslinked in a solution of 1-ethyl-3-(3-dimethylaminopropyl) carbodiimide and N-hydroxysuccinimide.

### Administration of Additional Therapeutic Agents

The methods disclosed herein can further comprise, or alternatively consist essentially of, or yet further consist of administration of an effective amount of additional therapeutic agents to augment or enhance the therapeutic efficacy of the disclosed methods. Non-limiting examples of additional therapeutic agents to augment or enhance the therapeutic efficacy of the disclosed methods include a therapeutic polypeptide such as osteoprotegerin, bone morphogenic protein (BMP), growth factors, IGF-I, IGF-II, platelet-derived growth factor, basic and acidic fibroblast growth factor (FGF), BMP2, BMP4, OP-1, FGF1, FGF2, TGF-γ1, TGF-β2, TGF-β3, Collagen 1, laminin 1-6, fibronectin, parathyroid hormone related peptide (PTHrP), vitronectin, etidronate, clodronate, alendronate, pamidronate, risedronate, zoledronate, hydroxyapatite, hyaluronic acid, prednisone, budesonide, prednisolone, cyclosporine, tacrolimus, sirolimus, everolimus, azathioprine, leflunomide, mycophenolate, abatacept, adalimumab, anakinra, certolizumab, etanercept, golimumab, infliximab, ixekizumab, natalizumab, rituximab, secukinumab, secukinumab, tocilizumab, ustekinumab, vedolizumab, basiliximab, daclizumab, muromonab, teriparatide and chitosan. The compositions may be supplemented with exogenous testosterone, dihydrotestosterone, estrogens, estradiol, GH/IGF-1, thyroid hormone, parathyroid hormone, calcitonin, glucocorticoids, cortisol and vitamin D.

The following example disclosure below is provided to illustrate but not limit the invention.

### EXAMPLE 1: STUDIES OF COMPOSITE MATERIALS COMPRISING MC-GAG COUPLED TO PHOSPHATE RESERVOIR MATERIALS

The current available materials and methods for the reconstruction of bone such as in skull defects have significant drawbacks in complications for patients as well as healthcare costs. Such limitations have demonstrated a need for new strategies, such as the development of targeted regenerative therapies that are safe and readily available during surgery. In this context, embodiments of the invention are focused on the development of a surgically practical, off-the-shelf, biomaterials-based regenerative solution for the treatment of skull defects.

In surgeries entering the skull for etiologies including trauma, stroke, cancer, and congenital anomalies, skull reconstruction is required to prevent neurologic sequelae, cerebral protection, and potential psychosocial burdens particularly in large defects. The current clinically available materials for cranial defect reconstruction are limited by donor site morbidity for autologous bone and complications and cost for alloplastic materials, thereby providing an opportunity to develop strategies targeting skull regeneration. Despite decades of research, contemporary regenerative strategies consisting of expanded stem cells and growth factor cocktails delivered by scaffolding materials have not attained clinical translation secondary to the drawbacks of surgical impracticality, cost, time consumption, and the untoward effects of supraphysiologic, uncompensated activities of growth factors. With the increasing understanding of the instructive capabilities of the extracellular matrix, we previously demonstrated the potential for an extracellular matrix-inspired, synthetic material composed of nanoparticulate mineralized collagen glycosaminoglycan (MC-GAG) to serve as a materials-only regenerative strategy. MC-GAG was capable of partially regenerating calvarial defects in vivo without the addition of pre-expanded progenitor cells or exogenous growth factor supplementation. Mechanistically, we recently demonstrated that the intrinsic mineral content on MC-GAG generates a temporospatial spike in phosphate ion equilibrium within the local microenvironment that is essential for its effects on osteoprogenitor cell differentiation via the activities of the type III sodium-phosphate cotransporters, SLC20A1/PiT-1 and SLC20A2/PiT-2. From our studies, we have determined that a composite materials consisting of MC-GAG with a phosphate eluting hydrogel (MCGPh) will augment osteogenic differentiation of progenitor cells. 2. MCGPh materials will exceed MC-GAG in in vivo rabbit skull healing.

Our previous work established the essential and stimulatory role of phosphate ion content on MC-GAG materials for inducing in vitro osteogenic differentiation and in vivo bone healing. However, phosphate elution is a time-limited event on MC-GAG and limited by the species of nanoparticulate mineral present in the material. Our preliminary data has indicated a novel composite material combining a phosphate-eluting hydrogel to MC-GAG (MCGPh) extended the period of time for phosphate ion elution beyond that of MC-GAG. Our studies provide evidence that the MCGPh will augment osteoprogenitor cell differentiation and mineralization as compared to MC-GAG.

Our established in vivo rabbit calvarial defect data has indicated that MC-GAG may be a potentially useful starting material to develop a cell-free, off-the-shelf, materials-only strategy for calvarial regeneration. However, the efficacy of the MC-GAG base material for in vivo skull healing is limited to at most 60% that of native calvarium by mineralization as well as biomechanical testing. Thus, improvement of MC-GAG is necessary for clinical translation. Given the essential role of phosphate ion elution from MC-GAG on osteogenic differentiation and our studies on this, we believe that such properties can be harnessed to improve in vivo calvarial regeneration. We can also evaluate in vivo bone healing, biomechanics, inflammation, vascularization, and local and systemic safety of MCGPh composite scaffolds for calvarial regeneration.

Osseous defects of the skull occur secondary to trauma, stroke, cerebral aneurysms, cancer, and congenital craniofacial anomalies (5-10). Cranioplasty, or calvarial reconstruction, is indicated for cerebral protection, neurologic symptoms (11, 12), psychological, social, and vocational reasons. Although cranioplasties are common procedures in craniofacial surgery, the current available clinical materials have significant shortcomings. While autologous bone is limited by size and donor site morbidity (13), alloplastic materials are plagued with complications such as extrusion, high cost, and infection with 5-12 times more complications than autologous bone depending on report (6, 7, 14-16). The drawbacks of the available clinical materials provide an opportunity to develop methods that specifically target calvarial bone regeneration.

Despite decades of study, regenerative therapies have failed to truly enter the arena of surgical standard-of-care. Thus, the question must be asked whether the traditional strategy of integrating scaffolding material with progenitor cells supplemented with growth factors will ultimately be translatable. For regeneration to be applicable to patients, three criteria must be satisfied from a surgical standpoint: 1. Regenerative therapies must be safe; 2. Regenerative therapies must be relatively simple; 3. Regenerative therapies must be practical. For the first criterion, one major difficulty in adopting supraphysiologic dosages of growth factors for bone regeneration is the lack of safety. Within the spinal fusion literature, bone morphogenetic protein (BMP)-2 use has decreased since 2007 due to complications including heterotopic ossification, osteolysis, and pain (17, 18). For the second and third criteria, regenerative therapies that require more complexity than currently available reconstructive options will not likely achieve translation. For calvarial regeneration, the concept of progenitor cell harvest, ex vivo expansion, and reimplantation is far greater in complexity and more costly than autologous bone grafting or alloplastic materials. Thus, the lack of simplicity results in a surgically impractical therapy.

Embodiments of the invention include a cell-free, materials only approach to calvarial regeneration using lessons from biology. The observation that the unique compositions of the extracellular matrix (ECM) can differentially instruct cell fate determination has led to a significant interest in generating ECM-inspired regenerative materials (3, 19-39). These observations provide evidence that each unique ECM-inspired material generates a new local microenvironment that requires mechanistic understanding of cell physiology and material interactions. Our laboratory has characterized a novel nanoparticulate mineralized collagen glycosaminoglycan (MC-GAG) material for skull regeneration. Our work has generated significant data on the osteogenic signaling mechanisms induced by the material on osteoprogenitors as well as the ability for MC-GAG to heal in vivo rabbit calvarial defects without growth factors or ex vivo expanded progenitor cells (3, 4, 28), suggesting that MC-GAG may be a promising base material for a cell-free, “off-the-shelf”, materials only solution for calvarial regeneration. However, the development of MC-GAG is still in its infancy in that the highest efficacy reached in regeneration is at most 50-60% of native calvarium. Thus, further improvements are clearly possible. We have recently noted that the temporospatial influence of MC-GAG on the microenvironmental phosphate ion equilibrium is an essential component to the osteogenic activities of MC-GAG on multipotent progenitor cells. (2) We have characterized the mechanism to require heterodimers of the type III sodium phosphate cotransporters, solute carrier 20A1/phosphate ion transporter-1 (SLC20A1/PiT-1) and SLC20A2/PiT-2. Our studies provide evidence that extending the amount of time for phosphate elution will augment the ability of MC-GAG to induce osteogenic differentiation and in vivo bone healing.

An ECM-based, materials-only approach for bone regeneration can eliminate the necessity of ex vivo progenitor cell expansion and growth factor supplementation. While delivery of mineral has been well-described, increases in mineral content significantly changes material properties. The material in this application overcomes this limitation with the delivery of an inorganic ion via a phosphate reservoir such as a nanoparticle or a water soluble hydrogel.

Clinically available options for cranioplasty can be categorized into autologous or alloplastic materials. Three types of autologous bone exist: banked orthotopic calvarial bone, fresh heterotopic bone graft, or fresh, vascularized heterotopic bone flap. Banked calvarial bone refers to bone removed at the time of decompressive craniectomies and stored sterilely for 2-3 months until resolution of intracranial hypertension. While the advantages of banked calvarial bone are the lack of an additional donor site and return of the exact tissue removed, failure due to resorption or infection ranges between 7-50% (40-43). Fresh autologous bone grafts harvested from adjacent uninvolved calvaria or other anatomical sites such as the iliac crest have a 5% rate of significant resorption and the lowest infection rates among available materials (41). Although superior to banked calvaria, autologous grafts are limited by donor site availability and morbidity. In hostile wound environments (radiation, infection, reoperation), vascularized bone flaps may be required due to their superiority over grafts (6, 7). Vascularized bone flaps denote bony tissue harvested with its vasculature intact for transfer as an autologous transplant and re-vascularization to distant vessels. As this procedure requires microsurgical plastic and reconstructive expertise, vascularized bone flaps are typically limited to tertiary care centers.

Unlike autologous bone, alloplastic materials, such as titanium, methylmethacrylate (MMA), hydroxy apatite (HA) bone cement, and polyetheretherketone (PEEK) incur no donor site morbidities, require less operative time, may be three-dimensionally printed for perfect adaptation, and have no size limitations (44, 45). However, alloplastic materials have the drawbacks of cost, extrusion, lack of re-vascularization, and infection (7). Compared to autologous bone, alloplastic materials are five times more likely to have infectious complications and failures requiring explantation range from 16-62% depending on the type of alloplast (14, 16, 46-49). Thus, despite the ease of use, alloplastic materials are significantly inferior to autologous bone.

### Regenerative Materials for Calvarial Reconstruction Inspired by the Extracellular Matrix

The limitations of cranioplasty materials combined with the clinical importance for calvarial reconstruction suggest a need for alternative approaches. Recently, the increasing understanding that extracellular matrix (ECM) compositions can instruct differential cell fate determination has generated heightened interest in developing ECM-inspired materials for regeneration (1, 50). For skeletal regeneration, the majority of such materials are composites based on the major organic component of bone, collagen I. While collagen alone has demonstrated little to no capabilities in calvarial regeneration without progenitor cells and/or growth factors (1, 28, 51), collagen I combined with glycosaminoglycans (Col-GAG) and/or mineral content demonstrate significantly improved osteogenic capabilities. GAGs are known to promote cell adhesion and organize extracellular signals by locally concentrating combinations of growth factors (37, 52-56). Mineral content in the form of calcium phosphate, the major inorganic component of bone, has known osteogenic capabilities via both ion-mediated and stiffness-mediated osteogenic signaling mechanisms (57-64). Calcium (Ca²⁺) and phosphate (PO₄³⁻ ) ions have been reported to upregulate transcription of osteogenic genes including BMP-2, Smad1/5/8, osteogenic transcription factors (Runx2, osterix), and the late osteogenic genes osteopontin (OPN) and osteocalcin (OCN) (57-60, 65-67). Both of the type III sodium phosphate symporters (SLC20A1/PiT-1 and SLC20A2/PiT-2) have been reported to induce hMSC osteogenic differentiation and potentiate growth factor-induced osteogenic differentiation (65, 68-71). Similar to other reports using osteoprogenitor cells (72), we have observed that high phosphate concentrations in the media induced primary bone marrow-derived human mesenchymal stem cells (hMSCs, CD105⁺CD166⁺CD29⁺CD44⁺CD14⁻CD34⁻CD45 to undergo osteogenic differentiation in a manner that could be abrogated by downregulation of sodium phosphate transporters. Primary hMSCs were cultured in growth media with a baseline phosphate concentration of 0.9 mM (Lo Pi), osteogenic growth media supplemented with 10 mM b-glycerophosphate, 0.1 mM dexamethasone, and 50 mg/mL of ascorbic acid (Hi Pi), or Hi Pi media with 1 mM of phosphonoformic acid (PFA), an inhibitor of sodium phosphate symporters, for 14 days and subjected to Alizarin Red staining to detect mineralization (2). PFA treatment alone prevented mineralization of hMSCs, despite the presence of other osteogenic stimulators such as dexamethasone. These data suggested that phosphate ion induced signaling or sensing is an obligate process for osteogenic differentiation.

Murine knockout models supported the importance of PiT-1 and PiT-2 in skeletal development. While PiT-1 knockouts are embryonic lethal, a murine model with hypomorphic PiT-1 alleles resulting in an 85% reduction in expression exhibited a smaller skeleton grossly and PiT-2 upregulation (73). Couasnay et al demonstrated that postnatal conditional chondrocytic PiT-1 ablation diminished growth plate maturation via chondrocyte apoptosis but did not affect mineralization (74). Unlike the embryonic lethality of PiT-1 knockout mice, PiT-2 knockout mice were viable but had impaired bone quality (68, 75). In combination, organic and inorganic components of bone ECM serve to organize a variety of osteogenic mechanisms to equate to a net osteogenic state.

### Nanoparticulate Mineralized Collagen Glycosaminoglycan Materials Induce Osteogenesis and In Vivo Calvarial Healing

Our group has characterized the osteogenic mechanisms and in vivo utility of a novel, synthetic material composed of nanoparticulate mineralized Col-GAG (MC-GAG) in calvarial regeneration (2-4, 28, 33, 34, 76-80). MC-GAG is an open cell foam with a hydrated, crosslinked elastic modulus of 3.9 kiloPascals (kPa) synthesized using a lyophilization technique called concurrent mapping (79-82). Unlike previous methods of mineral incorporation in collagen scaffolds using immersion which generated a coating of mineral, concurrent mapping allows for distribution of nanoparticulate mineral content throughout the collagen fibers, similar to cancellous bone. Elemental compositional studies using x-ray photoelectron spectroscopy (XPS) has determined that the mineral content of cell-free MC-GAG consists of a 1:1 calcium to phosphate ratio after synthesis (2).

Several unique aspects of MC-GAG suggest potential for clinical translation. First, cell-based mineralization on MC-GAG resembles the composition of physiological bone. Primary hMSCs were cultured on Col-GAG or MC-GAG and subjected to XPS to assess the changes in elemental composition of the materials. Mineral content found on Col-GAG demonstrated a calcium to phosphate ratio (Ca/P) of 2.5, whereas MC-GAG exhibited a ratio of 1.7-1.8. Based on the known compositions of calcium phosphate minerals, mineral content produced on MC-GAG after culture with hMSCs is similar to hydroxy apatite (Ca/P=1.7) (83), whereas Col-GAG Ca/P ratios after culture with hMSCs resembled amorphous calcium phosphate (Ca/P ranging from 1.3-2.5) (83). These data provide evidence that MC-GAG induced osteoprogenitor cells to mineralize in a manner that produced calcium phosphate species similar to physiological hydroxy apatite.

Another unique aspect of MC-GAG is the ability of the material to induce osteogenic differentiation in primary bone marrow-derived primary hMSCs independent of growth factors, such BMP-2 (3). When compared to non-mineralized Col-GAG as a control, hMSCs differentiated on MC-GAG in osteogenic growth medium containing 10 mM b-glycerol phosphate, 50 mg/mL ascorbic acid, and 0.1 mM dexamethasone expressed higher quantities of alkaline phosphatase (ALP), collagen I (Col I), and osteopontin (OPN) on quantitative reverse transcriptase polymerase chain reaction (QPCR), suggesting that the material itself is osteogenic. When untreated hMSCs were compared to BMP-2-treated hMSCs on MC-GAG, both micro-computed tomography (micro-CT) and Alizarin Red staining demonstrated no differences between the conditions. These data provide evidence that MC-GAG induced hMSCs to undergo osteogenic differentiation independent of growth factors.

The efficacy of MC-GAG in in vitro differentiation prompted us to evaluate the in vivo calvarial regenerative capabilities (4). New Zealand white rabbits (2-3 months old, n=4 per group) were divided into seven treatment groups: 1) defect without reconstruction, 2) Col-GAG scaffold only, 3) Col-GAG seeded with BMSCs ex vivo, 4) Col-GAG seeded with BMSCs and BMP-2 ex vivo, 5) MC-GAG scaffold only, 6) MC-GAG seeded with BMSCs ex vivo, 7) MC-GAG seeded with BMSCs and BMP-2 ex vivo. For groups with BMSCs, three weeks prior to creation of the skull defect, bone marrow was harvested and 2×10⁶ BMSCs were cultured on 14 mm Col-GAG and MC-GAG in osteogenic medium and untreated or treated with BMP-2 (50 ng/ml). Twelve weeks after implantation in 14 mm full thickness, extradural defects, calvaria were explanted and subjected to micro-CT and biomechanical testing. MC-GAG-reconstructed defects demonstrated higher mineralization that approached 50-60% of the surrounding native calvarium. Interestingly, empty MC-GAG scaffolds demonstrated no difference in mineralization compared to MC-GAG loaded with BMSCs or BMSCs/BMP-2. In contrast, empty Col-GAG and Col-GAG loaded with BMSCs demonstrated no statistically significant differences compared to unreconstructed defects. When loaded with BMSCs/BMP-2, Col-GAG-mediated mineralization improved.

Biomechanical properties of the regenerated calvaria was evaluated using reference point indentation (Biodent, Active Life Scientific, Santa Barbara, CA) paralleled the micro-CT data. Indentation data was analyzed for total indentation distance (TID), as a measure of toughness or resistance to microfracture, and unloading slope (US) of force (N) to displacement (mm) curves, as a measure of stiffness. Biomechanical data from each cranial defect was internally controlled with adjacent native calvaria and expressed as a ratio of regenerated bone/native bone. Similar to the micro-CT data, MC-GAG displayed higher microfracture resistance (inversely proportional to TID ratio) and stiffness (directly proportional to US ratio) compared to either the unreconstructed (defect only) or Col-GAG treatment groups, whereas Col-GAG did not demonstrate significant differences compared to the unreconstructed defect. In both measurements, cell-free MC-GAG-regenerated calvaria did not differ significantly from MC-GAG+BMSCs or BMSCs/BMP-2. These data provide evidence that MC-GAG induced healing reached up to 50-60% that of surrounding calvarium by both mineralization and biomechanical properties. Furthermore, MC-GAG induces in vivo calvarial healing independent of ex vivo expanded progenitor cells or exogenous BMP-2.

### PRELIMINARY STUDIES

### Nanoparticulate Mineralized Collagen Glycosaminoglycan Materials Effect Dynamic Changes in Phosphate Ion Equilibrium in the Local Microenvironment

Given the potential for phosphate ion to induce osteogenic differentiation, we first evaluated the effect of the intrinsic mineral content of MC-GAG on local phosphate concentrations. Cell-free Col-GAG and MC-GAG were cultured in growth media (baseline phosphate ion concentration of 0.9 mM) and phosphate concentrations were assessed over 21 days with media changes every 3 days. While Col-GAG maintained a soluble phosphate concentration identical to basal medium, MC-GAG displayed dynamic changes over time with indications of both early elution and late absorption. Using a repeated measures general linear model to evaluate the differences in phosphate concentrations between the scaffolds over time, there was a significant main effect of scaffold type [F(1,8)=15.004, p=0.005] as well as time [F(6,48)=63.636, p<0.001] on phosphate concentration with an estimated marginal mean of 0.92(95% CI 0.89-0.95) mM for Col-GAG and 1.08(95% CI 0.96-1.21) mM for MC-GAG. When cultured with hMSCs, a dynamic relationship with phosphate elution and absorption was again found with MC-GAG whereas Col-GAG remained stable with basal media phosphate concentrations. These data provide evidence that MC-GAG dynamically controls soluble phosphate in the surrounding media in a temporally-dependent manner with elution of phosphate early in culture followed by deposition in the absence or presence of hMSCs.

### Nanoparticulate Mineralized Collagen Glycosaminoglycan Materials Induces hMSC-mediated Mineralization in Low Phosphate Environments

To understand that significance of the alterations in phosphate ion equilibrium, we next evaluated the potential for osteogenic differentiation in the presence of MC-GAG in low versus high phosphate conditions. hMSCs were cultured on Col-GAG as a control material or MC-GAG for 8 weeks in media with basal phosphate concentrations (0.9 mM, Lo Pi) or high phosphate concentrations (10 mM, Hi Pi) and mineralization was assessed using both micro-CT as well as Alizarin Red staining (2). While mineralization on Col-GAG required the presence of high phosphate concentrations to demonstrate any measurable amount of mineralization, MC-GAG was capable of inducing mineralization in the presence of low or high phosphate conditions. Of note, when we quantified the amount of mineralization, MC-GAG in high phosphate conditions still induced greater amounts of mineralization compared to low phosphate conditions. For both materials and both phosphate concentrations, we evaluated whether PiT-1 and PiT-2 were involved in the differentiation process. Using 1 mM of PFA to inhibit the activities of PiT-1 and PiT-2, mineralization was found to be inhibited on both materials regardless of media phosphate concentrations.

These observations provide evidence three conclusions: 1. The intrinsic phosphate content within MC-GAG is sufficient for osteogenic differentiation and mineralization; 2. The sodium phosphate transporters are required for osteogenic differentiation and mineralization; 3. The addition of exogenous phosphate augmented the mineralization capabilities of MC-GAG.

### PiT-1 and PiT-2 Serve Non-redundant Roles in Inducing Osteogenic Gene Expression and Mineralization on MC-GAG

The inhibition of mineralization by PFA indicated that sodium phosphate transporters were important for MC-GAG-mediated differentiation, however the lack of specificity in PFA-mediated inhibition precluded the elucidation of the exact identities as PFA has been reported to affect both type II and type III transporters (84,85). Hence, we next evaluated the necessity of PiT-1, PiT-2, or the combination of PiT-1 and PiT-2 using RNA interference for MC-GAG activity given that the two isoforms are the dominant sodium phosphate transporters expressed in hMSCs and implicated in osteogenic differentiation.

50 pmol of small interfering RNA (siRNA) for PiT-1 (siPiT-1), PiT-2 (siPiT-2), PiT-1 and PiT-2 (siPiT-1/2), or scrambled control (siControl) were transiently transfected in 2.5×10⁴ hMSCs with Lipofectamine RNAiMAX (ThermoFisher, Waltham, MA). 24 hours after transfection, cells were seeded onto Col-GAG or MC-GAG materials and cultured for 7 days in basal media. Both gene and protein expression of PiT-1 were found to be elevated on MC-GAG materials compared to Col-GAG in siControl transfected hMSCs. PiT-1 expression was efficiently knocked down in the presence of siPiT-1 or siPiT-1/2 transfections at both the gene and protein expression levels. In siPiT-2 transfected cells, a compensatory increase in PiT-1 protein was found on western blot analysis, suggesting a compensatory effect. Unlike PiT-1, PiT-2 expression was not significantly different between siControl transfected hMSCs on the two scaffolds for either gene or protein expression. siPiT-2 and siPiT-1/2 transfection efficiently reduced both gene and protein expression of PiT-2 on either material. siPiT-1, however, demonstrated differences between the two scaffolds with Col-GAG showing a simultaneous reduction in PiT-2 protein expression compared to siControl. In contrast, siPIT-1 transfected hMSCs on MC-GAG showed an increase in PiT-2 expression compared to siControl transfected hMSCs.

To understand the role of PiT-1 and PiT-2 in mineralization on MC-GAG, osteogenic gene expression was evaluated in siPiT-1, siPiT-2, and siPiT1/2 transfected hMSCs differentiated on the two scaffolds for 7 days. For non-mineralized Col-GAG materials, siPiT-1, siPiT-2, and siPiT-1/2 transfections demonstrated no effect on any of the osteogenic markers tested, whereas both early (ALP) and late (OCN) osteogenic markers were reduced in hMSCs on MC-GAG.

With respect to mineralization assessed via micro-CT analysis after 8 weeks of culture, no significant quantitative differences were noted on non-mineralized Col-GAG materials in the presence of siPiT-1, siPiT-2, or siPiT-1/2. These quantitative data concurred with histologic analysis at 14 days of culture for mineralization using Alizarin red staining, albeit a minor qualitative reduction could be detected in the double knockdown cells on Col-GAG. In contrast, knockdown of PiT-1, PiT-2, or both transporters resulted in a decrease in mineralization quantitatively on micro-CT and qualitatively on Alizarin Red staining on mineralized MC-GAG scaffolds. Similar to the osteogenic gene expression, siPiT-1 and siPiT-2 individually as well as siPiT-1/2 demonstrated equivalent reductions in mineralization. The combination of the osteogenic gene expression and mineralization data provide evidence that PiT-1 and PiT-2 are both required for maximal osteogenic differentiation of hMSCs on mineralized MC-GAG materials in a non-redundant, yet non-additive manner.

### Synthesis of a Phosphate-Eluting Chitosan Hydrogel as a Potential MC-GAG Composite

As we demonstrated that phosphate ion from MC-GAG was critical for osteogenic signaling, we hypothesized that extending phosphate release may improve its activity. However, phosphate ion within the material exists in a mineral form, of which significant alterations would change the material properties. Hence, we next sought to synthesize a phosphate delivery carrier that could be added to MC-GAG.

Given the porous nature of MC-GAG, we first explored the potential of synthesizing a composite of MC-GAG and a water-soluble hydrogel (86, 87). Among hydrogels, chitosan (CS)-based hydrogels have been used as scaffolds for tissue engineering in the past two decades due to several advantageous properties: 1) high biodegradability and 2) easy structural modification (88). Additionally, the chemistry of chitosan as a natural cationic polyelectrolyte polymer with an amine group enables to synthesis of ionically cross-linked chitosan hydrogels (89). CS hydrogel prepared by a self-assembly, ionic crosslinking method using a low molecular weight anionic crosslinker such as tripolyphosphate (TPP), sodium sulfate or cyclodextrin (CD) has been reported to form gels controlled by the charge density of CS and oppositely charged molecules (88). We hypothesized that CS-based hydrogel in combination with β-glycerophosphate (b-GP) would result in sustainable phosphate eluting hydrogel and enhance osteogenesis compared to scaffold alone.

To determine the optimal formulation of a CS-based hydrogel with b-GP, we first evaluated the dosing of b-GP. 2% of CS was dissolved into 0.1 M hydrochloric acid and b-GP was dissolved in distilled water respectively. For the preparation of CS hydrogel, the b-GP solution was added dropwise to the CS solution and mixed in an ice bath to prevent gelation. After mixing 30 minutes, the pH of each mixture set to be 7.4. The obtained solution was incubated at 37 °C for hydrogel formation. Gross properties and time to solidification of the hydrogel formed by ionic crosslinking between CS and b-GP were then assessed (88). CS:b-GP (1:10) formed a hydrogel after more than 1 h incubation at 37 °C whereas CS:b-GP (1:20) formed hydrogel for 10 min, CS:b-GP (1:30) for 6 min CS:b-GP (1:40) for 3 min. These data provide evidence that the content of b-GP was directly related to the strength of crosslinking via ionic bonds between CS and b-GP, resulting in less incubation time to form hydrogel.

### CS/b-GP Hydrogel Induces Mineralization and Extends Phosphate-Release

To investigate whether CS/b-GP hydrogel induced mineralization, primary hMSCs were cultured on CS/b-GP (1:20) hydrogel or CS/b-GP (1:40) hydrogel in basal growth medium for 7 days or 14 days were stained with Alizarin Red. Both CS/b-GP (1:20) and CS/b-GP (1:40) demonstrated more mineralization at day 14 compared to day 7, albeit not reaching statistical significance. These data provide evidence that CS/b-GP has an intrinsic ability to induce mineralization of primary hMSCs.

To evaluate phosphate release behavior from CS/b-GP hydrogel, we performed colorimetric assays for phosphate release. Given that we found no significant differences between the two concentrations of CS/b-GP and that the 1:20 concentration appeared qualitatively better, we chose to continue the rest of our experiments using the 1:20 concentration. hMSCs were cultured on CS/b-GP hydrogel or without hydrogel (Control) in growth medium for 14 days. Prior to each media change every 3 days, a sample of media was collected and kept for the phosphate assays. Similar to MC-GAG, CS/b-GP also demonstrated an elution of phosphate early in culture when compared to control. These data provide evidence that CS/b-GP may potentially serve as a water-soluble delivery strategy for phosphate ions.

### A Composite of Nanoparticulate Mineralized Collagen Glycosaminoglycan and CS/b-GP Hydrogel Displays Extended Phosphate-Release

To investigate whether CS/B-GP hydrogel incorporates with MC-GAG and functions as a supporting phosphate delivery carrier, we synthesize a composite of MC-GAG and CS/b-GP (MCGPh). MC-GAG and CS/β-GP 1:20 were prepared as described above. Prior to solidification of CS/b-GP, MC-GAG was soaked in CS/β-GP solution for 2 h in an ice bath to evenly distribute the hydrogel throughout the porous architecture of MC-GAG. The composite material was then transferred to 37 °C and incubated for 20 min to solidify the hydrogel.

The gross stability of MCGPh in growth medium was first evaluated at day 1 and 7. Compared to MC-GAG alone, MCGPh grossly displayed a hydrogel appearance and was volumetrically larger from the solidified coating of the scaffold overall. At 7 days in growth medium, the structure was largely stable although a mild decrease in size was notable suggesting dissolution of a portion of the hydrogel.

In addition to stability, phosphate release behavior from MCGPh hydrogel was performed by the colorimetric assay for phosphate determination in the presence of hMSCs cultured on the two materials. hMSCs were cultured on MC-GAG and MCGPh hydrogel in growth medium for 7 days. Over the course of this period of time, media was collected prior to media change at every 3 days and subjected the phosphate colorimetric assay. Both materials displayed the characteristic peak of phosphate elution early in culture. However, compared to MC-GAG alone, MCGPh demonstrated a sustained and extended release of phosphate during the course of the week compared to MC-GAG alone. These data provide evidence that MCGPh can serve as an extended-release version of MC-GAG.

We have demonstrated that a novel biomaterial inspired by the extracellular matrix of bone activates osteogenic differentiation of primary human mesenchymal stem cells and regenerates massive calvarial defects in rabbits without the addition of ex vivo expanded progenitor cells or exogenous growth factors. Although these data provide evidence that MC-GAG may hold promise as a materials-only, off-the-shelf, point-of-care solution for skull defects, improvement in efficacy must be accomplished. Two lines of evidence provide evidence that phosphate ion may serve as a potential target for improving MC-GAG. First, we have characterized the intrinsic MC-GAG content to be necessary and sufficient for hMSC osteogenic differentiation and mineralization. We further confirmed this finding with the characterization of the mechanism of action to be dependent upon heterodimers of the PiT-1 and PiT-2 sodium phosphate cotransporters. Second, despite the sufficiency of the intrinsic mineral content in activating differentiation and mineralization, exogenous phosphate via the addition of higher concentrations of b-glycerophosphate in the media surpassed the mineralization capabilities of MC-GAG alone. Thus, our studies indicate that a composite material that increases the amount of time or quantity of phosphate elution from MC-GAG would step closer to clinical translation. In our data, we have described the synthesis of MC-GAG combined with a phosphate eluting hydrogel (MCGPh).

### Assessing the Effect of a Phosphate Eluting Hydrogel on Osteogenesis Induced by Nanoparticulate Mineralized Collagen Glycosaminoglycan Materials

The efficacy of MC-GAG in stimulating osteogenic differentiation, matrix mineralization, and ultimately in vivo calvarial healing depends significantly on its phosphate content as downregulation of the sodium phosphate symporters via both a small molecule inhibitor as well as knockdown experiments result in a dramatic inhibition of osteogenic differentiation and mineralization of hMSCs. Given that the phosphate elution effects of MC-GAG are temporally limited, we hypothesize that extending the timing of phosphate release on MC-GAG via the composite MCGPh material will result in an augmentation of osteogenic differentiation and mineralization.

### Illustrative Experimental Approaches

Comparison of MCGPh and MC-GAG on hMSC Expression of Osteogenic Differentiation Markers.

Primary hMSCs from three different donors can be used for each experiment. 2×10⁵ hMSCs can be seeded on Col-GAG (non-mineralized, negative control material), MC-GAG, or MCGPh in growth medium. At 0, 3, 7, 14, and 28 days of culture, gene and protein expression analyses can be performed. Total RNA extracted using the RNeasy system (Qiagen, Valencia, CA) can be subjected to QPCR for expression of osteogenic genes including early markers (ALP, Col I) and later markers (OCN, OPN, and BSP2).

### Comparison of MCGPh and MC-GAG on Activation of Intracellular Signaling Pathways Related to Osteogenic Differentiation.

We have previously characterized that a downregulation of PiT-1 and/or PiT-2 resulted in a decrease in ERK1/2 phosphorylation with a reciprocal increase in p-Smad1/5 phosphorylation. If this pathway is affected by phosphate eluted from MCGPh, we would anticipate that p-ERK1/2 would increase and p-Smad1/5 would decrease compared to MC-GAG. To test this, hMSCs cultured in the identical manner as described above for the same timepoints can be used. Total protein from each of the materials can be extracted with Phosphosafe lysis buffer (Novagen, Madison, WI), subjected to 4-20% SDS-PAGE (Biorad, Hercules, CA), and blotted for phosphorylated and total ERK1/2, phosphorylated and total Smad1/5, phosphorylated and total p38 as a control, and b-actin (phosphoprotein antibodies from Cell Signaling Technologies, all other antibodies from Santa Cruz Biotechnology, Santa Cruz, CA).

### Comparison of MCGPh and MC-GAG on Matrix Deposition and Mineralization.

At 8 weeks of culture, mineralized matrix deposition can be assessed using microscopy and micro-CT scanning. Scaffolds can be fixed in 10% formalin and scanned at medium resolution settings with a source voltage of 70 E (kVp) and I (mA) of 114. Two-dimensional (2D) images can be analyzed to establish volumes of interest (Scanco Image Processing Language version 5.6). Optimum arbitrary threshold values of 20 (showing scaffold and mineralization) and 80 (mineralization alone) can be used to quantify percent mineralized volume. Histomorphometric analysis of three-dimensional (3D) reconstructions can be performed using Scanco Evaluation scripts no. 2 (3D segmentation of two volumes of interest: solid dense in transparent low-density object) for 3D images and script no. 6 (bone volume/density only bone evaluation) for volume determinations. Following scanning, scaffolds can be embedded in paraffin and sectioned at 4 mm in thickness and stained with H&E and Alizarin Red to evaluate cellular content and mineralized matrix deposition, respectively. All assays can be performed at least in triplicate with three different hMSC donors and analyzed with analysis of variance (ANOVA) with posthoc comparisons under the Tukey criterion (SPSS Version 24, Chicago, IL) presuming normality of distribution. Should any of the data be non-normally distributed, a Kruskal-Wallis test with posthoc comparisons using the Bonferroni adjustment can be employed.

The disclosure provided herein allows artisans to observe the similarities and differences in osteogenic gene expression and signaling mechanisms induced by MCGPh compared to that induced by MC-GAG in primary human mesenchymal stem cells. MCGPh may increase the amount, length of time, or both for osteogenic gene expression. MCGPh will likely signal via ERK-1/2 phosphorylation in an extended fashion. MCGPh will likely increase the quantity of mineralization found and potentially induce earlier mineralization in culture.

We have previously demonstrated that the stiffness of MC-GAG is tied to its osteogenic abilities such that significantly softer materials have a reduced ability for osteogenic differentiation (79, 80). If we detect a reduction in osteogenic differentiation, we can address this first by generating a panel of hydrogels of varying stiffness. In our preliminary data, we have demonstrated that the relative ratio of b-GP changes the properties of the chitosan/b-GP hydrogel such that higher quantities of b-GP would allow for stiffer hydrogels. Alternatively, we can also increase the stiffness of the MC-GAG base material with a variation in the relative amounts of EDC/NHS used for crosslinking.

Evaluating the in Vivo Skull Regenerative Effects of a Composite Material Combining a Phosphate Eluting Hydrogel and Nanoparticulate Mineralized Collagen glycosaminoglycan.

Our in vivo rabbit calvarial defect data established that MC-GAG may be a potentially useful base material in a cell-free, off-the-shelf, materials-only strategy for calvarial regeneration provided that efficacy may be improved. Our preliminary data has confirmed the positive effects of phosphate on MC-GAG function. Thus, we hypothesize that MCGPh would improve in vivo calvarial regeneration. In this context, we can evaluate in vivo bone healing, biomechanics, inflammation, vascularization, and local and systemic safety of MCGPh for calvarial regeneration.

### Experimental Approach

Reconstruction of Critical-Sized Rabbit Cranial Defects with MCGPh

To observe the efficacy of MCGPh in calvarial regeneration, we can evaluate healing at 3 months in 14 mm rabbit calvarial defects. Three types of defect reconstruction can be evaluated: 1) unreconstructed, defect only negative control 2) MC-GAG control 3) MCGPh.

Based on our published studies, the predicted differences in scaffold mineralization ranges from 26% (negative control) to 50% (MC-GAG reconstructed defects) with a standard deviation of 15%. We have determined that the n in each of the 3 treatment groups will need to have at least 6 viable data points (alpha=0.05; beta=0.05, effect size=1.1 for a power of 0.97). Calculating for an attrition rate of 10%, the total number of rabbits necessary can be 20 rabbits total.

Female and male New Zealand White rabbits (2-3 months old, 3 of each) can be injected subcutaneously with enrofloxacin (5 mg/kg) and acepromazine (1 mg/kg). The rabbit can be placed until anesthesia with isoflurane gas (1.5-3%) and pain can be controlled with subcutaneous buprenorphine 0.05 mg/kg and carprofen 4 mg/kg. Following skin incision and subperiosteal dissection, a 14 mm full thickness, extradural craniotomy defect can be created and the bone can be lifted away without injury to the dura (90). Each calvarium can be treated with one of the 8 different types of defect reconstruction described above.

### Biomechanical Properties of Regenerated Bone in Massive Cranial Defects.

Freshly explanted skulls can be characterized for biomechanical properties using the BioDent reference point indentation device (Active Life Scientific) according to manufacturer's instructions. Indentations can be conducted in five areas throughout the regenerate and five areas within the native bone at a force of 2N, an indentation frequency of 2 Hz, and 10 indentation cycles at a touchdown force of 0.1 N using probe assembly type BP2. Indentation data can be analyzed with the BioDent software for the total indentation distance (TID), first cycle indentation distance (ID1st), loading slope (LS), and unloading slopes (US). Toughness, or resistance to fracture, can be determined by TID and ID1st. Relative stiffness can be determined by LS and US. To minimize differences in the thickness of bone for each animal as well as the bone healing capabilities, data from each cranial defect can be internally controlled with the native calvarial bone.

### Mineralization of Regenerated Bone in Massive Cranial Defects.

Following biomechanical testing, the explanted skulls can be fixed in 10% formalin and subjected to micro-CT to quantitatively determine the regenerated bone volume. Scans can be performed using medium resolution settings with a source voltage of 70 E (kVp) and I (μA) of 114. 2D images can be analyzed to establish volumes of interest (Scanco Image Processing Language version 5.6). Optimum arbitrary threshold values of 20 (showing scaffold and mineralization) and 80 (mineralization alone) can be used to quantify percent mineralized volume. Histomorphometric analysis 3D reconstructions can be performed using Scanco Evaluation scripts no. 2 (3D segmentation of two volumes of interest: solid dense in transparent low-density object) for 3D images and script no. 6 (bone volume/density only bone evaluation) for volume determinations. Additionally, the densities of regenerated bone with relationship to native calvarium can be determined with Hounsfield Units (HU) using Osirix (Pixmeo SARL, Bermex, Switzerland). The ratio of mean density of defect/mean density of native bone can be calculated for each condition to account for individual differences in rabbit bone.

### Local and Systemic Safety of Mcgph Implantation.

Local and downstream tissue assessment can be performed on the dura and scalp. Both tissue types can be harvested at the time of calvarial explantation and histologic analysis with H&E staining can be performed to qualitatively assess inflammation, foreign body reaction, fibrosis, and vascularity of the areas immediately adjacent to the regenerated bone compared to the areas adjacent to native calvarium only. To ensure objectivity, trained pathologists blinded to the treatment groups can be used for qualitative analysis. All animals can be sent for necropsy by trained veterinary pathologists for the purposes of determining safety and documenting systemic effects both grossly and histologically.

We do not anticipate difficulties in establishment of the animal model due to our multi-year experience using the exact same model for our proof-of-concept experiments as well as multiple publications from our laboratory. We can readily generate quantitative data on bone healing and the strength of bone produced by MCGPh compared to MC-GAG at 3 months following implantation. Our data indicates that the results will demonstrate improved healing and biomechanical strength of the regenerate in the MCGPh composite scaffolds compared to MC-GAG. We do not anticipate any toxicities of MCGPh given our previous data with MC-GAG.

In summary, we have demonstrated the potential of delivering phosphate to selected sites using both hydrogels and nanoparticles and have synthesized both materials in the laboratory. Preferred embodiments of the invention include hydrogel phosphate delivery in the form of a composite with MC-GAG as well as in the form of composite MC-GAG carrying phosphate-eluting nanoparticles.

### REFERENCES

1. Lee J C, Volpicelli E J. Bioinspired Collagen Scaffolds in Cranial Bone Regeneration: From Bedside to Bench. Adv Healthc Mater. 2017. Epub Jun. 6, 2017. doi: 10.1002/adhm.201700232. PubMed PMID: 28585295.
2. Ren X, Zhou Q, Bedar M, Foulad D, Huang K X, Dejam D, et al. Modulating Temporospatial Phosphate Equilibrium by Nanoparticulate Mineralized Collagen Materials Induces Osteogenesis via PiT-1 and PiT-2. Advanced Healthcare Materials. 2023: Under Review.
3. Ren X, Bischoff D, Weisgerber D W, Lewis M S, Tu V, Yamaguchi DT, et al. Osteogenesis on nanoparticulate mineralized collagen scaffolds via autogenous activation of the canonical BMP receptor signaling pathway. Biomaterials. 2015; 50:107-14. doi: 10.1016/j.biomaterials.2015.01.059. PubMed PMID: 25736501; PubMed Central PMCID: PMC4364277.
4. Ren X, Tu V, Bischoff D, Weisgerber D W, Lewis M S, Yamaguchi D T, et al. Nanoparticulate mineralized collagen scaffolds induce in vivo bone regeneration independent of progenitor cell loading or exogenous growth factor stimulation. Biomaterials. 2016; 89:67-78. doi: 10.1016/j.biomaterials.2016.02.020. PubMed PMID: 26950166.
5. Lew T A, Walker J A, Wenke J C, Blackbourne L H, Hale R G. Characterization of craniomaxillofacial battle injuries sustained by United States service members in the current conflicts of Iraq and Afghanistan. J Oral Maxillofac Surg. 2010; 68(1):3-7. doi: 10.1016/j.joms.2009.06.006. PubMed PMID: 20006147.
6. Fong A J, Lemelman B T, Lam S, Kleiber G M, Reid R R, Gottlieb L J. Reconstructive approach to hostile cranioplasty: A review of the University of Chicago experience. J Plast Reconstr Aesthet Surg. 2015; 68(8):1036-43. doi: 10.1016/j.bjps.2015.04.014. PubMed PMID: 25971417.
7. Lee J C, Kleiber G M, Pelletier A T, Reid R R, Gottlieb L J. Autologous immediate cranioplasty with vascularized bone in high-risk composite cranial defects. Plast Reconstr Surg. 2013; 132(4):967-75. doi: 10.1097/PRS.0b013e31829f4b59. PubMed PMID: 24076686.
8. Chao A H, Yu P, Skoracki R J, Demonte F, Hanasono M M. Microsurgical reconstruction of composite scalp and calvarial defects in patients with cancer: A 10-year experience. Head Neck. 2012. doi: 10.1002/hed.21999. PubMed PMID: 22331614.
9. Lee E I, Chao A H, Skoracki R J, Yu P, DeMonte F, Hanasono M M. Outcomes of calvarial reconstruction in cancer patients. Plast Reconstr Surg. 2014; 133(3):675-82. doi: 10.1097/01.prs.0000438061.46290.33. PubMed PMID: 24263391.
10. Tessier P. Autogenous bone grafts taken from the calvarium for facial and cranial applications. Clin Plast Surg. 1982; 9(4):531-8. PubMed PMID: 6756759.
11. Fodstad H, Love J A, Ekstedt J, Fridén H, Liliequist B. Effect of cranioplasty on cerebrospinal fluid hydrodynamics in patients with the syndrome of the trephined. Acta Neurochir (Wien). 1984; 70(1-2):21-30. PubMed PMID: 6741628.
12. Hagan M, Bradley J P. Syndrome of the Trephined: Functional Improvement After Reconstruction of Large Cranial Vault Defects. J Craniofac Surg. 2017; 28(5):1129-30. doi: 10.1097/SCS.0000000000003747. PubMed PMID: 28582309.
13. Banwart J C, Asher M A, Hassanein R S. Iliac crest bone graft harvest donor site morbidity. A statistical evaluation. Spine (Phila Pa 1976). 1995; 20(9):1055-60. PubMed PMID: 7631235.
14. Fearon J A, Griner D, Ditthakasem K, Herbert M. Autogenous Bone Reconstruction of Large Secondary Skull Defects. Plast Reconstr Surg. 2017; 139(2):427-38. doi: 10.1097/PRS.0000000000002941. PubMed PMID: 28125534.
15. Blum K S, Schneider S J, Rosenthal A D. Methyl methacrylate cranioplasty in children: long-term results. Pediatr Neurosurg. 1997; 26(1):33-5. PubMed PMID: 9361115.
16. Afifi A, Djohan R S, Hammert W, Papay F A, Barnett A E, Zins J E. Lessons learned reconstructing complex scalp defects using free flaps and a cranioplasty in one stage. J Craniofac Surg. 2010; 21(4):1205-9. Epub Jul. 9, 2010. doi: 10.1097/SCS.0b013e3181e17c1e. PubMed PMID: 20613618.
17. Cahill K S, Chi J H, Day A, Claus E B. Prevalence, complications, and hospital charges associated with use of bone-morphogenetic proteins in spinal fusion procedures. JAMA. 2009; 302(1):58-66. doi: 10.1001/jama.2009.956. PubMed PMID: 19567440.
18. Lord E L, Cohen J R, Buser Z, Meisel H J, Brodke D S, Yoon S T, et al. Trends, Costs, and Complications of Anterior Cervical Discectomy and Fusion With and Without Bone Morphogenetic Protein in the United States Medicare Population. Global Spine J. 2017; 7(7):603-8. Epub Aug. 17, 2017. doi: 10.1177/2192568217699207. PubMed PMID: 28989837; PubMed Central PMCID: PMC 5624371.
19. Caliari S R, Ramirez M A, Harley B A. The development of collagen-GAG scaffold-membrane composites for tendon tissue engineering. Biomaterials. 2011; 32(34):8990-8. doi: 10.1016/j.biomaterials.2011.08.035. PubMed PMID: 21880362; PubMed Central PMCID: PMC 3947519.
20. Caliari S R, Harley B A. The effect of anisotropic collagen-GAG scaffolds and growth factor supplementation on tendon cell recruitment, alignment, and metabolic activity. Biomaterials. 2011; 32(23):5330-40. doi: 10.1016/j.biomaterials.2011.04.021. PubMed PMID: 21550653; PubMed Central PMCID: PMC 3947515.
21. Caliari S R, Weisgerber D W, Ramirez M A, Kelkhoff D O, Harley B A. The influence of collagen-glycosaminoglycan scaffold relative density and microstructural anisotropy on tenocyte bioactivity and transcriptomic stability. J Mech Behav Biomed Mater. 2012; 11:27-40. doi: 10.1016/j.jmbbm.2011.12.004. PubMed PMID: 22658152; PubMed Central PMCID: PMC 3947516.
22. Caliari S R, Mozdzen L C, Armitage O, Oyen M L, Harley B A. Award Winner in the Young Investigator Category, 2014 Society for Biomaterials Annual Meeting and Exposition, Denver, Colorado, Apr. 16-19, 2014: Periodically perforated core-shell collagen biomaterials balance cell infiltration, bioactivity, and mechanical properties. J Biomed Mater Res A. 2014; 102(4):917-27. doi: 10.1002/jbm.a.35058. PubMed PMID: 24327556.
23. Caliari S R, Gonnerman E A, Grier W K, Weisgerber D W, Banks J M, Alsop A J, et al. Collagen Scaffold Arrays for Combinatorial Screening of Biophysical and Biochemical Regulators of Cell Behavior. Adv Healthc Mater. 2014. doi: 10.1002/adhm.201400252. PubMed PMID: 24989480.
24. Caliari S R, Harley B A. Collagen-GAG Scaffold Biophysical Properties Bias MSC Lineage Choice in the Presence of Mixed Soluble Signals. Tissue Eng Part A. 2014. doi: 10.1089/ten.TEA.2013.0400. PubMed PMID: 24568607.
25. Caliari S R, Harley B A. Structural and Biochemical Modification of a Collagen Scaffold to Selectively Enhance MSC Tenogenic, Chondrogenic, and Osteogenic Differentiation. Adv Healthc Mater. 2014. doi: 10.1002/adhm.201300646. PubMed PMID: 24574180.
26. Harley B A, Leung J H, Silva E C, Gibson L J. Mechanical characterization of collagen-glycosaminoglycan scaffolds. Acta Biomater. 2007; 3(4):463-74. doi: 10.1016/j. actbio.2006.12.009. PubMed PMID: 17349829.
27. Harley B A, Lynn A K, Wissner-Gross Z, Bonfield W, Yannas I V, Gibson L J. Design of a multiphase osteochondral scaffold. II. Fabrication of a mineralized collagen-glycosaminoglycan scaffold. J Biomed Mater Res A. 2010; 92(3):1066-77. doi: 10.1002/jbm.a.32361. PubMed PMID: 19301274.
28. Lee J C, Pereira C T, Ren X, Huang W, Bischoff D, Weisgerber D W, et al. Optimizing Collagen Scaffolds for Bone Engineering: Effects of Cross-linking and Mineral Content on Structural Contraction and Osteogenesis. J Craniofac Surg. 2015. doi: 10.1097/SCS.0000000000001918. PubMed PMID: 26147021.
29. Lynn A K, Best S M, Cameron R E, Harley B A, Yannas I V, Gibson L J, et al. Design of a multiphase osteochondral scaffold. I. Control of chemical composition. J Biomed Mater Res A. 2010; 92(3):1057-65. doi: 10.1002/jbm.a.32415. PubMed PMID: 19301264.
30. Pence J C, Gonnerman E A, Bailey R C, Harley B A. Strategies to balance covalent and non-covalent biomolecule attachment within collagen-GAG biomaterials. Biomater Sci. 2014; 2(9):1296-304. doi: 10.1039/C4BM00193A. PubMed PMID: 25147727; PubMed Central PMCID: PMC 4136535.
31. Ren X, Weisgerber D W, Bischoff D, Lewis M S, Reid R R, He T C, et al. Nanoparticulate Mineralized Collagen Scaffolds and BMP-9 Induce a Long-Term Bone Cartilage Construct in Human Mesenchymal Stem Cells. Adv Healthc Mater. 2016; 5(14):1821-30. doi: 10.1002/adhm.201600187. PubMed PMID: 27275929.
32. Weisgerber D W, Kelkhoff D O, Caliari S R, Harley B A. The impact of discrete compartments of a multi-compartment collagen-GAG scaffold on overall construct biophysical properties. J Mech Behav Biomed Mater. 2013; 28:26-36. doi: 10.1016/j.jmbbm.2013.07.016. PubMed PMID: 23973610; PubMed Central PMCID: PMC3960919.
33. Weisgerber D W, Caliari S R, Harley B A. Selective addition of mineral into collagen scaffolds to enhance hMSC osteogenesis and matrix remodeling. Acta Biomaterialia2014.
34. Weisgerber D W, Caliari S R, Harley B A. Mineralized collagen scaffolds induce hMSC osteogenesis and matrix remodeling. Biomater Sci. 2015; 3(3):533-42. doi: 10.1039/C4BM00397G. PubMed PMID: 25937924; PubMed Central PMCID: PMC4412464.
35. Keogh M B, O'Brien F J, Daly J S. Substrate stiffness and contractile behaviour modulate the functional maturation of osteoblasts on a collagen-GAG scaffold. Acta Biomater. 2010; 6(11):4305-13. doi: 10.1016/j.actbio.2010.06.001. PubMed PMID: 20570642.
36. Lyons F G, Al-Munajjed A A, Kieran S M, Toner M E, Murphy C M, Duffy G P, et al. The healing of bony defects by cell-free collagen-based scaffolds compared to stem cell-seeded tissue engineered constructs. Biomaterials. 2010; 31(35):9232-43. Epub Sep. 22, 2010. doi: 10.1016/j.biomaterials.2010.08.056. PubMed PMID: 20863559.
37. Murphy C M, Matsiko A, Haugh M G, Gleeson J P, O'Brien F J. Mesenchymal stem cell fate is regulated by the composition and mechanical properties of collagen-glycosaminoglycan scaffolds. J Mech Behav Biomed Mater. 2012; 11:53-62. doi: 10.1016/j.jmbbm.2011.11.009. PubMed PMID: 22658154.
38. Neumann A J, Alini M, Archer C W, Stoddart M J. Chondrogenesis of human bone marrow-derived mesenchymal stem cells is modulated by complex mechanical stimulation and adenoviral-mediated overexpression of bone morphogenetic protein 2. Tissue Eng Part A. 2013; 19(11-12):1285-94. doi: 10.1089/ten.TEA.2012.0411. PubMed PMID: 23289669.
39. Raftery R M, Woods B, Marques A L, Moreira-Silva J, Silva T H, Cryan S A, et al. Multifunctional biomaterials from the sea: Assessing the effects of chitosan incorporation into collagen scaffolds on mechanical and biological functionality. Acta Biomater. 2016; 43:160-9. Epub Jul. 8, 2016. doi: 10.1016/j.actbio.2016.07.009. PubMed PMID: 27402181.
40. Grant G A, Jolley M, Ellenbogen R G, Roberts T S, Gruss J R, Loeser J D. Failure of autologous bone-assisted cranioplasty following decompressive craniectomy in children and adolescents. J Neurosurg. 2004; 100(2 Suppl Pediatrics):163-8. doi: 10.3171/ped.2004.100.2.0163. PubMed PMID: 14758944.
41. Rocque B G, Amancherla K, Lew S M, Lam S. Outcomes of cranioplasty following decompressive craniectomy in the pediatric population. J Neurosurg Pediatr. 2013; 12(2):120-5. Epub Jun. 21, 2013. doi: 10.3171/2013.4.PEDS12605. PubMed PMID: 23790219.
42. Yadla S, Campbell P G, Chitale R, Maltenfort M G, Jabbour P, Sharan A D. Effect of early surgery, material, and method of flap preservation on cranioplasty infections: a systematic review. Neurosurgery. 2011; 68(4):1124-9; discussion 30. doi: 10.1227/NEU.0b013e31820a5470. PubMed PMID: 21242830.
43. Corliss B, Gooldy T, Vaziri S, Kubilis P, Murad G, Fargen K. Complications After In Vivo and Ex Vivo Autologous Bone Flap Storage for Cranioplasty: A Comparative Analysis of the Literature. World Neurosurg. 2016; 96:510-5. Epub Sep. 16, 2016. doi: 10.1016/j.wneu.2016.09.025. PubMed PMID: 27647038.
44. Marchac D, Greensmith A. Long-term experience with methylmethacrylate cranioplasty in craniofacial surgery. J Plast Reconstr Aesthet Surg. 2008; 61(7):744-52; discussion 53. doi: S1748-6815(08)00120-4 [pii]10.1016/j.bjps.2007.10.055. PubMed PMID: 18474454.
45. O'Reilly E B, Barnett S, Madden C, Welch B, Mickey B, Rozen S. Computed-tomography modeled polyether ether ketone (PEEK) implants in revision cranioplasty. J Plast Reconstr Aesthet Surg. 2015; 68(3):329-38. doi: 10.1016/j.bjps.2014.11.001. PubMed PMID: 25541423.
46. Blum K S, Schneider S J, Rosenthal A D. Methyl methacrylate cranioplasty in children: long-term results. Pediatr Neurosurg. 1997; 26(1):33-5. PubMed PMID: 9361115.
47. Shonka D C, Potash A E, Jameson M J, Funk G F. Successful reconstruction of scalp and skull defects: lessons learned from a large series. Laryngoscope. 2011; 121(11):2305-12. doi: 10.1002/lary.22191. PubMed PMID: 22020883.
48. Zins J E, Moreira-Gonzalez A, Papay F A. Use of calcium-based bone cements in the repair of large, full-thickness cranial defects: a caution. Plast Reconstr Surg. 2007; 120(5):1332-42. doi: 00006534-200710000-00030 [pii]10.1097/01.prs.0000279557.29134.cd. PubMed PMID: 17898609.
49. Kshettry V R, Hardy S, Weil R J, Angelov L, Barnett G H. Immediate Titanium Cranioplasty after Debridement and Craniectomy for Post-Craniotomy Surgical Site Infection. Neurosurgery. 2011. doi: 10.1227/NEU.0b013e31822fef2c. PubMed PMID: 21808217.
50. Szpalski C, Wetterau M, Barr J, Warren S M. Bone tissue engineering: current strategies and techniques—part I: Scaffolds. Tissue Eng Part B Rev. 2012; 18(4):246-57. doi: 10.1089/ten.TEB.2011.0427. PubMed PMID: 22029448.
51. Kruger E A, Im D D, Bischoff D S, Pereira C T, Huang W, Rudkin G H, et al. In vitro mineralization of human mesenchymal stem cells on three-dimensional type I collagen versus PLGA scaffolds: a comparative analysis. Plast Reconstr Surg. 2011; 127(6):2301-11. doi: 10.1097/PRS.0b013e318213a004. PubMed PMID: 21617464.
52. Hudalla G A, Murphy W L. Biomaterials that regulate growth factor activity via bioinspired interactions. Adv Funct Mater. 2011; 21(10):1754-68. doi: 10.1002/adfm.201002468. PubMed PMID: 21921999; PubMed Central PMCID: PMC3171147.
53. Miguez P A, Terajima M, Nagaoka H, Mochida Y, Yamauchi M. Role of glycosaminoglycans of biglycan in BMP-2 signaling. Biochem Biophys Res Commun. 2011; 405(2):262-6. Epub Jan. 8, 2011. doi: 10.1016/j.bbrc.2011.01.022. PubMed PMID: 21219861; PubMed Central PMCID: PMC 3057115.
54. Gama C I, Tully S E, Sotogaku N, Clark P M, Rawat M, Vaidehi N, et al. Sulfation patterns of glycosaminoglycans encode molecular recognition and activity. Nat Chem Biol. 2006; 2(9):467-73. Epub Jul. 30, 2006. doi: 10.1038/nchembio810. PubMed PMID: 16878128.
55. Murphy C M, Duffy G P, Schindeler A, O'brien F J. Effect of collagen-glycosaminoglycan scaffold pore size on matrix mineralization and cellular behavior in different cell types. J Biomed Mater Res A. 2016; 104(1):291-304. Epub Oct. 1, 2015. doi: 10.1002/jbm.a.35567. PubMed PMID: 26386362.
56. Hortensius R A, Harley B A. The use of bioinspired alterations in the glycosaminoglycan content of collagen-GAG scaffolds to regulate cell activity. Biomaterials. 2013; 34(31):7645-52. Epub Jul. 17, 2013. doi: 10.1016/j.biomaterials.2013.06.056. PubMed PMID: 23871542; PubMed Central PMCID: PMC4090944.
57. Viti F, Landini M, Mezzelani A, Petecchia L, Milanesi L, Scaglione S. Osteogenic Differentiation of MSC through Calcium Signaling Activation: Transcriptomics and Functional Analysis. PLOS One. 2016; 11(2):e0148173. doi: 10.1371/journal.pone.0148173. PubMed PMID: 26828589; PubMed Central PMCID: PMC4734718.
58. Barradas A M, Fernandes H A, Groen N, Chai Y C, Schrooten J, van de Peppel J, et al. A calcium-induced signaling cascade leading to osteogenic differentiation of human bone marrow-derived mesenchymal stromal cells. Biomaterials. 2012; 33(11):3205-15. doi: 10.1016/j.biomaterials.2012.01.020. PubMed PMID: 22285104.
59. Rosen V. BMP2 signaling in bone development and repair. Cytokine Growth Factor Rev. 2009; 20(5-6):475-80. doi: 10.1016/j.cytogfr.2009.10.018. PubMed PMID: 19892583.
60. Dvorak M M, Siddiqua A, Ward D T, Carter D H, Dallas S L, Nemeth E F, et al. Physiological changes in extracellular calcium concentration directly control osteoblast function in the absence of calciotropic hormones. Proc Natl Acad Sci U S A. 2004; 101(14):5140-5. doi: 10.1073/pnas.0306141101. PubMed PMID: 15051872; PubMed Central PMCID: PMC387387.
61. Koori K, Maeda H, Fujii S, Tomokiyo A, Kawachi G, Hasegawa D, et al. The roles of calcium-sensing receptor and calcium channel in osteogenic differentiation of undifferentiated periodontal ligament cells. Cell Tissue Res. 2014; 357(3):707-18. Epub May 20, 2014. doi: 10.1007/s00441-014-1918-5. PubMed PMID: 24842051.
62. Wen L, Wang Y, Wang H, Kong L, Zhang L, Chen X, et al. L-type calcium channels play a crucial role in the proliferation and osteogenic differentiation of bone marrow mesenchymal stem cells. Biochem Biophys Res Commun. 2012; 424(3):439-45. doi: 10.1016/j.bbrc.2012.06.128. PubMed PMID: 22771798.
63. González-Vázquez A, Planell J A, Engel E. Extracellular calcium and CaSR drive osteoinduction in mesenchymal stromal cells. Acta Biomater. 2014; 10(6):2824-33. Epub Feb. 10, 2014. doi: 10.1016/j.actbio.2014.02.004. PubMed PMID: 24525034.
64. Mizumachi H, Yoshida S, Tomokiyo A, Hasegawa D, Hamano S, Yuda A, et al. Calcium-sensing receptor-ERK signaling promotes odontoblastic differentiation of human dental pulp cells. Bone. 2017; 101:191-201. Epub May 12, 2017. doi: 10.1016/j.bone.2017.05.012. PubMed PMID: 28506888.
65. Shih Y R, Hwang Y, Phadke A, Kang H, Hwang N S, Caro E J, et al. Calcium phosphate-bearing matrices induce osteogenic differentiation of stem cells through adenosine signaling. Proc Natl Acad Sci U S A. 2014; 111(3):990-5. doi: 10.1073/pnas. 1321717111. PubMed PMID: 24395775; PubMed Central PMCID: PMC3903237.
66. Beck G R, Jr., Zerler B, Moran E. Phosphate is a specific signal for induction of osteopontin gene expression. Proc Natl Acad Sci U S A. 2000; 97(15):8352-7. doi: 10.1073/pnas.140021997. PubMed PMID: 10890885; PubMed Central PMCID: PMC26951.
67. Guerrero F, Herencia C, Almaden Y, Martinez-Moreno J M, Montes de Oca A, Rodriguez-Ortiz M E, et al. TGF-beta prevents phosphate-induced osteogenesis through inhibition of BMP and Wnt/beta-catenin pathways. PLoS One. 2014:9(2):e89179. doi: 10.1371/journal.pone.0089179. PubMed PMID: 24586576; PubMed Central PMCID: PMC3937350.
68. Yamada S, Wallingford M C, Borgeia S, Cox T C, Giachelli C M. Loss of PiT-2 results in abnormal bone development and decreased bone mineral density and length in mice. Biochem Biophys Res Commun. 2018; 495(1):553-9. Epub Nov. 11, 2017. doi: 10.1016/j.bbrc.2017.11.071. PubMed PMID: 29133259; PubMed Central PMCID: PMC5739526.
69. Yoshiko Y, Candeliere G A, Maeda N, Aubin J E. Osteoblast autonomous Pi regulation via Pit1 plays a role in bone mineralization. Mol Cell Biol. 2007; 27(12):4465-74. doi: 10.1128/MCB.00104-07. PubMed PMID: 17438129; PubMed Central PMCID: PMC1900051.
70. Carroll S H, Wigner N A, Kulkarni N, Johnston-Cox H, Gerstenfeld L C, Ravid K. A2B adenosine receptor promotes mesenchymal stem cell differentiation to osteoblasts and bone formation in vivo. J Biol Chem. 2012; 287(19):15718-27. doi: 10.1074/jbc.M112.344994. PubMed PMID: 22403399; PubMed Central PMCID: PMC3346096.
71. Suzuki A, Ghayor C, Guicheux J, Magne D, Quillard S, Kakita A, et al. Enhanced expression of the inorganic phosphate transporter Pit-1 is involved in BMP-2-induced matrix mineralization in osteoblast-like cells. J Bone Miner Res. 2006; 21(5):674-83. doi: 10.1359/jbmr.020603. PubMed PMID: 16734382.
72. Katsumata Y, Kajiya H, Okabe K, Fukushima T, Ikebe T. A salmon DNA scaffold promotes osteogenesis through activation of sodium-dependent phosphate cotransporters. Biochem Biophys Res Commun. 2015; 468(4):622-8. Epub Nov. 10, 2015. doi: 10.1016/j.bbrc.2015.10.172. PubMed PMID: 26551467.
73. Bourgine A, Pilet P, Diouani S, Sourice S, Lesoeur J, Beck-Cormier S, et al. Mice with hypomorphic expression of the sodium-phosphate cotransporter PiT1/Slc20a1 have an unexpected normal bone mineralization. PLOS One. 2013; 8(6):e65979. Epub Jun. 13, 2013. doi: 10.1371/journal.pone.0065979. PubMed PMID: 23785462; PubMed Central PMCID: PMC 3681848.
74. Couasnay G, Bon N, Devignes C S, Sourice S, Bianchi A, Véziers J, et al. PiT1/Slc20a1 Is Required for Endoplasmic Reticulum Homeostasis, Chondrocyte Survival, and Skeletal Development. J Bone Miner Res. 2019; 34(2):387-98. Epub Nov. 20, 2018. doi: 10.1002/jbmr.3609. PubMed PMID: 30347511.
75. Beck-Cormier S, Lelliott C J, Logan J G, Lafont D T, Merametdjian L, Leitch V D, et al. Slc20a2, Encoding the Phosphate Transporter PiT2, Is an Important Genetic Determinant of Bone Quality and Strength. J Bone Miner Res. 2019; 34(6):1101-14. Epub Mar. 19, 2019. doi: 10.1002/jbmr.3691. PubMed PMID: 30721528; PubMed Central PMCID: PMC6618161.
76. Ren X, Zhou Q, Foulad D, Dewey M J, Bischoff D, Miller T A, et al. Nanoparticulate mineralized collagen glycosaminoglycan materials directly and indirectly inhibit osteoclastogenesis and osteoclast activation. J Tissue Eng Regen Med. 2019; 13(5):823-34. Epub Apr. 15, 2019. doi: 10.1002/term.2834. PubMed PMID: 30803152; PubMed Central PMCID: PMC6529242.
77. Ren X, Zhou Q, Foulad D, Tiffany A S, Dewey M J, Bischoff D, et al. Osteoprotegerin reduces osteoclast resorption activity without affecting osteogenesis on nanoparticulate mineralized collagen scaffolds. Sci Adv. 2019; 5(6):eaaw4991. Epub Jun. 12, 2019/. doi: 10.1126/sciadv.aaw4991. PubMed PMID: 31206025; PubMed Central PMCID: PMC6561746.
78. Zhou Q, Ren X, Bischoff D, Weisgerber D W, Yamaguchi D T, Miller T A, et al. Nonmineralized and Mineralized Collagen Scaffolds Induce Differential Osteogenic Signaling Pathways in Human Mesenchymal Stem Cells. Adv Healthc Mater. 2017; 6(23). Epub Sep. 25, 2017. doi: 10.1002/adhm.201700641. PubMed PMID: 28945007.
79. Zhou Q, Lyu S, Bertrand A A, Hu A C, Chan C H, Ren X, et al. Stiffness of Nanoparticulate Mineralized Collagen Scaffolds Triggers Osteogenesis via Mechanotransduction and Canonical Wnt Signaling. Macromol Biosci. 2020:e2000370. Epub Dec. 31, 2020. doi: 10.1002/mabi.202000370. PubMed PMID: 33382197.
80. Zhou Q, Ren X, Oberoi M K, Bedar M, Caprini R M, Dewey M J, et al. β-Catenin Limits Osteogenesis on Regenerative Materials in a Stiffness-Dependent Manner. Adv Healthc Mater. 2021; 10(23):e2101467. Epub Oct. 8, 2021. doi: 10.1002/adhm.202101467. PubMed PMID: 34585526; PubMed Central PMCID: PMC8665088.
81. Harley B A, Lynn A K, Wissner-Gross Z, Bonfield W, Yannas I V, Gibson L J. Design of a multiphase osteochondral scaffold II: fabrication of a mineralized collagen-GAG scaffold. J Biomed Mater Res A. 2010; 92(3):1066-77. Epub Mar. 21, 2009. doi: 10.1002/jbm.a.32361. PubMed PMID: 19301274.
82. Kanungo B P, Silva E, Van Vliet K, Gibson L J. Characterization of mineralized collagen-glycosaminoglycan scaffolds for bone regeneration. Acta Biomater. 2008; 4(3):490-503. Epub Jan. 26, 2008. doi: 10.1016/j.actbio.2008.01.003. PubMed PMID: 18294943.
83. Basu B, Katti D S, Kumar A. Advanced Biomaterials: Fundamentals, Processing, and Applications. Hoboken, New Jersey: John Wiley and Sons; 2009. 746 p.
84. Villa-Bellosta R, Bogaert Y E, Levi M, Sorribas V. Characterization of phosphate transport in rat vascular smooth muscle cells: implications for vascular calcification. Arterioscler Thromb Vasc Biol. 2007; 27(5):1030-6. Epub Feb. 22, 2007. doi: 10.1161/ATVBAHA.106.132266. PubMed PMID: 17322102.
85. Villa-Bellosta R, Sorribas V. Role of rat sodium/phosphate cotransporters in the cell membrane transport of arsenate. Toxicol Appl Pharmacol. 2008; 232(1):125-34. Epub Jun. 10, 2008. doi: 10.1016/j.taap.2008.05.026. PubMed PMID: 18586044.
86. Li H, Koenig A M, Sloan P, Leipzig N D. In vivo assessment of guided neural stem cell differentiation in growth factor immobilized chitosan-based hydrogel scaffolds. Biomaterials. 2014; 35(33):9049-57. Epub Aug. 10, 2014. doi: 10.1016/j.biomaterials.2014.07.038. PubMed PMID: 25112933.
87. Zhao Y, Zhang X, Wang Y, Wu Z, An J, Lu Z, et al. In situ cross-linked polysaccharide hydrogel as extracellular matrix mimics for antibiotics delivery. Carbohydr Polym. 2014; 105:63-9. Epub Jan. 29, 2014. doi: 10.1016/j.carbpol.2014.01.068. PubMed PMID: 24708953.
88. Ahmadi F, Oveisi Z, Samani S M, Amoozgar Z. Chitosan based hydrogels: characteristics and pharmaceutical applications. Res Pharm Sci. 2015; 10(1):1-16. PubMed PMID: 26430453; PubMed Central PMCID: PMC4578208.
89. Martinez-Ruvalcaba A. Viscoelastic properties of dispersed chitosan/xanthan hydrogels. Carbohydrate polymers. 2007; 67(4):586-95. doi: info:doi/.
90. Ashby E R, Rudkin G H, Ishida K, Miller T A. Evaluation of a novel osteogenic factor, bone cell stimulating substance, in a rabbit cranial defect model. Plast Reconstr Surg. 1996; 98(3):420-6. PubMed PMID: 8700975.

### CONCLUSION

This concludes the description of embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A composition of matter comprising a collagen glycosaminoglycan scaffold coupled to a phosphate reservoir material.

2. The composition of claim 1, wherein the collagen glycosaminoglycan scaffold is a nanoparticulate mineralized collagen glycosaminoglycan (MC-GAG) scaffold.

3. The composition of claim 1, wherein the phosphate reservoir material comprises a hydrogel coating disposed on the collagen glycosaminoglycan scaffold, wherein the hydrogel comprises phosphate moieties coupled thereto.

4. The composition of claim 3, wherein the hydrogel comprises a chitosan and β-glycerophosphate.

5. The composition of claim 1, wherein the phosphate reservoir material comprises nanoparticles.

6. The composition of claim 5, wherein the nanoparticles comprise a polyethylene glycol polymers combined with a beta-glycerophosphate.

7. The composition of claim 6, wherein the nanoparticles exhibit an average diameter of 200 nm to 600 nm.

8. The composition of claim 1, further comprising a therapeutic agent.

9. The composition of claim 8, wherein the therapeutic agent comprises a polypeptide.

10. The composition of claim 1, further comprising a pharmaceutical carrier.

11. A method of preparing a composition, comprising contacting a selected collagen glycosaminoglycan scaffold with a selected phosphate reservoir material such that the phosphate reservoir material is in operable contact the collagen glycosaminoglycan scaffold.

12. The method of claim 11, wherein the phosphate reservoir material comprises nanoparticles formed from polyethylene glycol polymers combined with a beta-glycerophosphate.

13. The method of claim 12, wherein the nanoparticles exhibit an average diameter of 200 nm to 600 nm

14. The method of claim 11, wherein the phosphate reservoir material comprises a hydrogel formed from a chitosan combined with a β-glycerophosphate.

15. A method of promoting osteogenesis in a subject in need thereof, comprising:
administering to the subject an effective amount of the composition of claim 1.
